(19) United States
(12) Patent Application Publication
(10) Pub. No.: US 2011/0288052 A1
(43) Pub. Date: Nov. 24, 2011

(54) NOVEL COMPOUNDS, PHARMACEUTICAL COMPOSITIONS CONTAINING SAME, AND METHODS OF USE FOR SAME

(76) Inventors: Craig A. Townsend, Baltimore, MD (US); Kandasamy Subburaj, Baltimore, MD (US); Jill Marie McFadden, Chapel Hill, NC (US)

(21) Appl. No.: 12/995,663

(22) PCT Filed: Jun. 2, 2009
(86) PCT No.: PCT/US2009/045945

§ 371 Date: Aug. 2, 2011

Related U.S. Application Data
(60) Provisional application No. 61/129,044, filed on Jun. 2, 2008.
(60) Provisional application No. 61/193,127, filed on Oct. 30, 2008.

(51) Int. Cl. C07D 333/32; C07D 409/12; C07D 413/12; A61K 31/5377; A61P 31/00; A61K 31/496; A61K 31/4535; A61P 35/04; A61P 3/04; A61K 31/381; A61K 31/69

(52) U.S. Cl. 514/64; 549/66; 514/445; 514/444; 549/60; 549/59; 544/146; 514/231.5; 549/4; 544/379; 514/252.13; 514/326; 546/213

(57) ABSTRACT

The class compounds of the present invention may be represented by Formula (I), wherein X may be O, S, or N. R¹ and R² are independently either H, C₁-C₂₀ alkyl, cycloalkyl, alkenyl, aryl, arylalkyl, or alkylaryl. R³ and R⁴ are independently either H, an aryl group, a heteroaryl group, and a heterocyclic ring group having 4 to 6 carbon atoms, wherein the aryl, heteroaryl, and heterocyclic moieties are optionally substituted with one or more of a first substitution group defined herein. In a further embodiment, R³ and R⁴ along with the atoms and bonds to which they are attached, form an optionally substituted 5-7 membered ring having at least one nitrogen atom within the ring structure.

### PRIORITY FILING

This application claims priority from U.S. Provisional Application No. 61/129,044, which was filed on Jun. 2, 2008 and is incorporated herein by reference, and U.S. Provisional Application No. 61/193,127, which was filed on Oct. 30, 2008 and is incorporated herein by reference.

### FIELD OF THE INVENTION

The present invention relates to novel compounds, pharmaceutical compositions containing the same, and methods of use for the inhibiting the fatty acid synthesis pathway by targeting the enzyme fatty acid synthase (FAS). Such compounds, compositions, and methods have a variety of therapeutically valuable uses including, but not limited to, treating cancerous cells which express or overexpress the FAS gene, treating obesity and treating invasive microorganisms which express or overexpress the FAS gene or a homolog thereof.

### BACKGROUND OF THE INVENTION

It is well known that new compounds for fighting cancer are needed. Compounds which are used as drugs used for chemotherapy must meet various criteria. First, they must be sufficiently cytotoxic and sufficiently non-toxic to non-cancerous cells. They must also be processible and bioavailable. On an unrelated front, new compounds to assist with the treatment of metabolic diseases and related conditions (like obesity) are also needed. Finally, new compounds to assist with the treatment of invasive microorganisms are also needed. The instant invention presents compounds useful for each of these applications by targeting fatty acid synthetic pathway, which is found within each targeted cell type.

Fatty acids have three primary roles in the physiology of cells. First, they are the building blocks of biological membranes. Second, fatty acid derivatives serve as hormones and intracellular messengers. Third, and of particular importance to the present invention, fatty acids are fuel molecules that can be stored in adipose tissue as triacylglycerols, which are also known as neutral fats.

There are four primary enzymes involved in the fatty acid synthetic pathway, fatty acid synthase (FAS), alkynyl CoA carboxylase (ACC), malic enzyme, and citric lyase. The principal enzyme, FAS, catalyzes the NADPH-dependent condensation of the precursors malonyl-CoA and alkynyl-CoA to produce fatty acids. NADPH is a reducing agent that generally serves as the essential electron donor at two points in the reaction cycle of FAS. The other three enzymes (i.e., ACC, malic enzyme, and citric lyase) produce the necessary precursors. Other enzymes, for example the enzymes that produce NADPH, are also involved in fatty acid synthesis.

Of the four enzymes in the fatty acid synthetic pathway, FAS is the preferred target for inhibition because it acts only within the pathway to fatty acids, while the other three enzymes are implicated in other cellular functions. Therefore, inhibition of one of the other three enzymes is more likely to affect normal cells.

FAS has an Enzyme Commission (E.C.) No. 2.3.1.85 and is also known as fatty acid synthetase, fatty acid ligase, as well as its systematic name acyl-CoA: malonyl-CoA C-acyltransferase (decarboxylating, oxoacyl- and enoyl-reducing and thioester-hydrolysing). There are seven distinct enzymes- or catalytic domains-involved in the FAS catalyzed synthesis of fatty acids: alkynyl transacylase, malonyl transacylase, beta-ketoacyl synthetase (condensing enzyme), beta-ketoacyl reductase, beta-hydroxyacyl dehydrase, enoyl reductase, and thioesterase. (Wakil, S. J., Biochemistry, 28: 4523-4530, 1989). All seven of these enzymes collectively form FAS.

Of the seven enzymatic steps carried out by FAS, the step catalyzed by the condensing enzyme (i.e., beta-ketoacyl synthetase) and the enoyl reductase have been the most common candidates for inhibitors that reduce or stop fatty acid synthesis. The condensing enzyme of the FAS complex is well characterized in terms of structure and function. The active site of the condensing enzyme contains a critical cysteine thiol, which is the target of antilipidemic reagents, such as, for example, the inhibitor cerulenin.

FAS inhibitors can be identified by the ability of a compound to inhibit the enzymatic activity of purified FAS. FAS activity can be assayed by numerous means known in the art, such as, for example, measuring the oxidation of NADPH in the presence of malonyl CoA (Dils, R. and Carey, E. M., “Fatty acid synthase from rabbit mammary gland,” Methods Enzymol, 35: 74-83, 1975). Other information relating to determination of whether a compound is an FAS inhibitor may be found in U.S. Pat. No. 5,981,575, the disclosure of which is hereby incorporated by reference.

Known inhibitors of the condensing enzyme include a wide range of chemical compounds, including alkylating agents, oxidants, and reagents capable of undergoing disulphide exchange. The binding pocket of the enzyme prefers long chain, E, E, dienes. In principal then, a reagent containing the sidechain diene and a group which exhibits reactivity with thiolate anions could be a good inhibitor of the condensing enzyme. Cerulenin [(2S,3R)-2,3-epoxy-4-oxo-7,10 dodecadienoyl amide] is an example of such a compound and has the following structure:

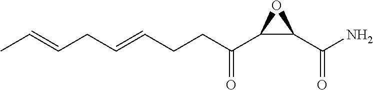

Cerulenin covalently binds to the critical cysteine thiol group in the active site of the condensing enzyme of fatty acid synthase, inactivating this key enzymatic step (Funabashi, et al., J. Biochem., 105: 751-755, 1989). While cerulenin has been noted to possess other activities, these either occur in microorganisms which may not be relevant models of human cells (e.g., inhibition of cholesterol synthesis in fungi, Omura (1976), Bacteriol. Rev., 40: 681-697; or diminished RNA synthesis in viruses, Perez, et al. (1991), FEBS, 280: 129-133), occur at a substantially higher drug concentrations (inhibition of viral HIV protease at 5 mg/ml, Moelling, et al. (1990), FEBS, 261: 373-377) or may be the direct result of the inhibition of endogenous fatty acid synthesis (inhibition of antigen processing in B lymphocytes and macrophages, Falo, et al. (1987), J. Immunol., 139: 3918-3923). Some data suggest that cerulenin does not specifically inhibit myristoylation of proteins (Simon, et al., J. Biol. Chem., 267: 3922-3931, 1992).

Various other compounds have been shown to inhibit fatty acid synthase (FAS). FAS inhibitors can be identified by the ability of a compound to inhibit the enzymatic activity of purified FAS. FAS activity can be assayed by measuring the incorporation of radiolabeled precursor (i.e., alkynyl-CoA or malonyl-CoA) into fatty acids or by spectrophotometrically measuring the oxidation of NADPH. (Dils, et al., Methods Enzymol., 35: 74-83). Preferably, inhibitors according to this invention will exhibit a suitable therapeutic index, safety profile, as well as efficacy, by showing IC₅₀ for FAS inhibition that is lower than the LD₅₀; more preferably LD₅₀ is at least an order of magnitude higher than IC₅₀.

Table 1, set forth below, lists several FAS inhibitors that are known in the art.

TABLE 1
Representative Inhibitors Of The Enzymes Of The Fatty Acid Synthesis Pathway

| | |
|---|---|
| Inhibitors of Fatty Acid Synthase | |
| 1,3-dibromopropanone | cerulenin |
| Ellman's reagent (5,5′-dithiobis(2-nitrobenzoic | phenyocerulenin |
| acid), DTNB) | melarsoprol |
| 4-(4′-chlorobenzyloxy) benzyl nicotinate (KCD- | iodoacetate |
| 232) | phenylarsineoxide |
| 4-(4′-chlorobenzyloxy) benzoic acid (MII) | pentostam |
| 2(5(4-chlorophenyl)pentyl)oxirane-2-carboxylate | melittin |
| (POCA) and its CoA derivative | thiolactomycin |
| ethoxyformic anhydride | |

Inhibitors for citrate lyase
(−) hydroxycitrate
(R,S)-S-(3,4-dicarboxy-3-hydroxy-3-methyl-
butyl)-CoA
S-carboxymethyl-CoA
Inhibitors for malic enzyme
periodate-oxidized 3-aminopyridine adenine
dinucleotide phosphate
5,5′-dithiobis(2-nitrobenzoic acid)
p-hydroxymercuribenzoate
N-ethylmaleimide
oxalyl thiol esters such as S-oxalylglutathione
gossypol
phenylglyoxal
2,3-butanedione
bromopyruvate
pregnenolone

| | |
|---|---|
| Inhibitors for alkynyl CoA carboxylase | |
| sethoxydim | 9-decenyl-1-pentenedioic acid |
| haloxyfop and its CoA ester | decanyl-2-pentenedioic acid |
| diclofop and its CoA ester | decanyl-l-pentenedioic acid |
| clethodim | (S)-ibuprofenyl-CoA |
| alloxydim | (R)-ibuprofenyl-CoA |
| trifop | fluazifop and its CoA ester |
| clofibric acid | clofop |
| 2,4-D mecoprop | 5-(tetradecycloxy)-2-furoic acid |
| dalapon | beta, beta′-tetramethylhexadecanedioic acid |
| 2-alkyl glutarate | tralkoxydim |
| 2-tetadecanylglutarate (TDG) | free or monothioester of beta, beta prime-methyl- |
| 2-oetylglutaric acid | substituted hexadecanedioic acid (MEDICA |
| N6,02-dibutyryl adenosine cyclic 3′,5′- | 16) |
| monophosphate | alpha-cyanco-4-hydroxycinnamate |
| N2,02-dibutyryl guanosine cyclic 3′,5′- | S-(4-bromo-2,3-dioxobutyl)-CoA |
| monophosphate | p-hydroxymercuribenzoate (PHMB) |
| CoA derivative of 5-(tetradecyloxy)-2-furoic | N6,02-dibutyryl adenosine cyclic 3′,5′- |
| acid (TOFA) | monophosphate |
| 2,3,7,8-tetrachlorodibenzo-p-dioxin | |

FAS inhibitors are also disclosed in U.S. patent application Ser. No. 08/096,908 and its CIP filed Jan. 24, 1994, the disclosures of which are hereby incorporated by reference. Included are inhibitors of fatty acid synthase, citrate lyase, CoA carboxylase, and malic enzyme.

Tomoda and colleagues (Tomoda et. al., Biochem. Biophys. Act 921: 595-598 1987; Omura el. al., J. Antibiotics 39: 1211-1218 1986) also describe Triacsin C (sometimes termed WS-1228A), a naturally occurring acyl-CoA synthetase inhibitor, which is a product of Streptomyces sp. SK-1894. The chemical structure of Triacsin C is 1-hydroxy-3-(E,E,E-2′,4′,7′-undecatrienylidine) triazene. Triacsin C causes 50% inhibition of rat liver acyl-CoA synthetase at 8. 7 uM; a related compound, Triacsin A, inhibits acyl CoA-synthetase by a mechanism which is competitive with long-chain fatty acids. Inhibition of acyl-CoA synthetase is toxic to animal cells. Tomoda et al. (Tomoda el. al., J. Biol. Chem. 266: 4214-4219, 1991) further teaches that Triacsin C causes growth inhibition in Raji cells, and have also been shown to inhibit growth of Vero and Hela cells. Tomoda el. al. also teaches that acyl-CoA synthetase is essential in animal cells and that inhibition of the enzyme has lethal effects.

Gamma-substituted-alpha-methylene-beta-carboxy-gamma-butyrolactones were disclosed in U.S. Pat. Nos. 5,981,575 and 5,759,837 (the disclosures of which are hereby incorporated by reference) as inhibitors of fatty acid synthesis, which can be used to inhibit growth of tumor cells by systematically reducing adipocyte mass and induce weight loss. These compounds were further disclosed as having the following advantages over the natural product cerulenin for therapeutic applications: (1) they do not contain the highly reactive epoxide group of cerulenin, (2) they are stable and soluble in aqueous solution, (3) they can be produced by a two-step synthetic reaction and thus easily produced in large quantities, and (4) they are easily tritiated to high specific activity for biochemical and pharmacological analyses.

Novel classes of thiophenes useful as FAS inhibitors are also disclosed in PCT Application Publication No. WO 2004/005277, the disclosure of which is incorporated by reference, as having the following generic structure.

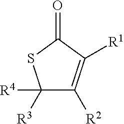

In each of the exemplified compounds, however, the R² position is limited to a certain subset of embodiments none of which overlaps with or disclose the compounds in the instant application.

Novel classes of thiophenes useful for FAS inhibition are also disclosed in PCT Application Publication No. WO 2008/057585, the disclosure of which is incorporated by reference, as having the same formula as above. Again, none of the exemplified compounds overlap with or otherwise disclose the compounds of the instant application, particularly at the R² position.

Other classes of novel compounds for use as FAS inhibitors are disclosed within PCT Application Publication Nos. WO 2007/014249; WO 2007/014247; WO 2005/117590; WO 2004/006835. Again, these applications do not disclose or exemplify any of the compounds disclosed below.

Accordingly, the instant invention addresses a need in the art for novel compounds useful as FAS inhibitors, which may be used to treat FAS expressing carcinomas, to treat obesity, or to treat microbial infections.

### SUMMARY OF THE INVENTION

The present invention relates to novel compounds useful as FAS inhibitors. To this end, the novel compounds of the present invention inhibit one or more of the enzymatic steps of fatty acid synthesis. Such compounds have a variety of therapeutically valuable uses including, but not limited to, treating cancerous cells which express or overexpress the FAS gene, treating obesity and treating invasive microorganisms which express or overexpress the FAS gene or a homolog thereof.

The class compounds of the present invention may be represented by Formula I:

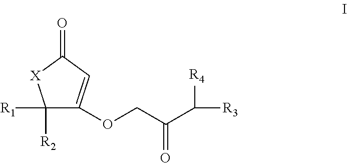

wherein X is comprised of a heteroatom which may be selected from any one of O, S, or N. R¹ and R² are independently selected from H, C₁-C₂₀ alkyl, cycloalkyl, alkenyl, aryl, arylalkyl, or alkylaryl. R³ and R⁴ are independently either a hydrogen atom or are members of a substituted or unsubstituted ring having 4-6 carbon atoms. In one embodiment, R³ and R⁴ are not both hydrogens. In another embodiment if neither R³ and R⁴ is a hydrogen, then they together form an optionally substituted ring structure having 4-6 carbon atoms. In further embodiments, R³ is a hydrogen and R⁴ is comprised of an aryl group, a heteroaryl group, or a heterocyclic ring group having 4 to 6 carbon atoms any of which are optionally substituted with one or more of a halogen atom, a C₁-C₃ alkyl group, a C₁-C₃ haloalkyl group, —OR⁵—SR⁵—CN, —CONH₂, —SO₂NH₂, —C(O)OR⁶—CONHR⁷ or a 5- or 6-membered cycloalkyl or heterocyclic ring. The latter 5- or 6-membered cycloalkyl or heterocyclic ring is optionally aromatic, optionally fused to adjacent atoms of R⁴, and/or is optionally substituted with R⁵.

R⁵ is comprised of any one of a C₁-C₈ alkyl, C₁-C₈ alkoxy, aryl, alkylaryl, arylalkyl, which may be optionally substituted with one or more halogen atoms, C₁-C₃ alkyl groups, C₁-C₃ alkoxy groups, C₁-C₃ haloalkyl groups, or C₁-C₃ haloalkoxy groups. R⁶ is comprised of a C₁-C₈ alkyl group. R⁷ is comprised of a C₁-C₈ alkyl, allyl group, a morpholine, a piperazine, an N-substituted piperazine with R⁵, or a 5- or 6-membered heterocycle containing N, O, S or any combination thereof.

In a further embodiment, R³ and R⁴ along with the atoms and bonds to which they are attached, form a 5-7 membered ring having at least one nitrogen atom within the ring structure, which is optionally substituted with one or more substitution groups defined herein.

Based on the foregoing, one or more compounds of the present invention, either alone or in combination with another active ingredient, may be synthesized and administered as a therapeutic composition using dosage forms and routes of administration contemplated herein or otherwise known in the art. Dosaging and duration will further depend upon the factors provided herein and those ordinarily considered by one of skill in the art. To this end, determination of a therapeutically effective amounts are well within the capabilities of those skilled in the art, especially in light of the detailed disclosure and examples provided herein.

### DESCRIPTION OF THE FIGURES

FIG. 1 illustrates one embodiment of a method of manufacturing the compounds of the instant invention, particularly C31.

FIG. 2 illustrates the replacement step of the process in FIG. 1 for the manufacture of the compound, C157.

FIG. 3 illustrates one embodiment for the method of preparing S enantiomers of the compounds of the present invention, particularly C 31.

FIG. 4 illustrates one embodiment for the method of preparing R enantiomers of the compounds of the present invention, particularly C 31.

FIG. 5 illustrates an alternative embodiment of a method of manufacturing the compounds of the instant invention, particularly C31.

FIG. 6 illustrates an alternative method of purifying the compounds of the present invention.

### DETAILED DESCRIPTION OF THE INVENTION

### Definitions

As used herein, “an alkyl group” denotes both straight and branched carbon chains with one or more carbon atoms, but reference to an individual radical such as “propyl” embraces only the straight chain radical, a branched chain isomer such as “isopropyl” specifically referring to only the branched chain radical.

As used herein, “substituted alkyl” is an alkyl group, as defined above, wherein one or more hydrogens of the alkyl group are substituted with 1 or more substituent groups as otherwise defined herein.

As used herein, “haloalkyl” refers to an alkyl group, as defined above, wherein one or more hydrogens of the alkyl group are substituted with 1 or more halogen atoms.

As used herein, “an alkoxy group” refers to a group of the formula alkyl-O—, where alkyl is as defined herein.

As used herein, “substituted alkoxy” refers to a substituted alkyl-O— group wherein the alkyl group is substituted as defined above.

As used herein, “haloalkoxy” refers to an alkoxy group, as defined above, wherein one or more hydrogens of the alkyl group are substituted with 1 or more halogen atoms.

As used herein, “alkenyl” refers to a saturated or unsaturated alkyl group, as defined herein, containing one or more carbon to carbon double bonds.

As used herein, “an aryl group” denotes a structure derived from an aromatic ring containing only carbon atoms. Examples include, but are not limited to a phenyl or benzyl radical and derivatives thereof.

As used herein, “arylalkyl” denotes an aryl group having one or more alkyl groups not at the point of attachment of the aryl group.

As used herein, “alkylaryl” denotes an aryl group having an alkyl group at the point of attachment.

As used herein, “heteroaryl” encompasses a monocyclic aromatic ring containing five or six ring atoms consisting of carbon and at least one non-carbon atom, which may be but is not limited to one or more of the following: nitrogen, oxygen, sulfur, phosphorus, boron, chlorine, bromine, or iodine.

As used herein, “heterocyclic” refers to a monovalent saturated or partially unsaturated cyclic non-aromatic carbon ring group which contains at least one heteroatom, in certain embodiments between 1 to 4 heteroatoms, which may be but is not limited to one or more of the following: nitrogen, oxygen, sulfur, phosphorus, boron, chlorine, bromine, or iodine. In further non-limiting embodiments, the heterocyclic ring may be comprised of between 1 and 10 carbon atoms.

As used herein, “cycloalkyl” refers to a monovalent or polycyclic saturated or partially unsaturated cyclic non-aromatic group containing all carbon atoms in the ring structure, which may be substituted with one or more substituent groups defined herein. In certain non-limiting embodiments the number of carbons comprising the cycloalkyl group may be between 3 and 7.

The present invention relates to a new class of compounds that are useful to inhibit the enzyme activity of the FAS protein, thus, inhibiting one or more of the enzymatic steps of fatty acid synthesis. Such compounds have a variety of therapeutically valuable uses including, but not limited to, treating cancerous cells which express or overexpress the FAS gene, treating obesity and treating invasive microorganisms which express or overexpress the FAS gene or a homolog thereof.

In one embodiment, the class compounds of the present invention may be represented by Formula I:

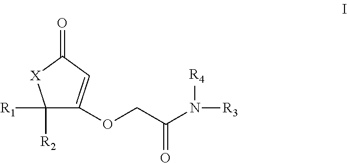

wherein X is comprised of a heteroatom which may be selected from any one of O, S, or N. R¹ and R² are independently selected from H, C₁-C₂₀ alkyl, cycloalkyl, alkenyl, aryl, arylalkyl, or alkylaryl. R³ and R⁴ are independently either a hydrogen atom or are members of a substituted or unsubstituted ring having 4-6 carbon atoms. In one embodiment, R³ and R⁴ are not both hydrogens. In another embodiment, if neither R³ and R⁴ is a hydrogen, then they together form an optionally substituted ring structure having 4-6 carbon atoms.

In further embodiments R³ is comprised of a hydrogen and R⁴ is comprised of a hydrogen, aryl group, a heteroaryl group, or a heterocyclic ring group having 4 to 6 carbon atoms wherein ring moiety of R⁴ is optionally substituted with one or more of a halogen atom, a C₁-C₃ alkyl group, a C₁-C₃ haloalkyl group, —OR⁵—SR⁵—CN, —CONH₂, —SO₂NH₂, —C(O)OR⁶, —CONHR⁷ or a 5- or 6-membered cycloalkyl or heterocyclic ring. The latter 5- or 6-membered cycloalkyl or heterocyclic ring is optionally aromatic, optionally fused to two adjacent atoms of R⁴, and/or is optionally substituted with one or more R⁵ substitutent groups.

In an alternative embodiment, and as discussed in greater detail below, R³ and R⁴ together, along with the atoms and bonds to which they are attached, form a 5-7 membered heterocyclic ring having at least one nitrogen atom within the ring structure.

R⁵ is comprised of any one of a C₁-C₈ alkyl, C₁-C₈ alkoxy, aryl, alkylaryl, arylalkyl, which may be optionally substituted with one or more halogen atoms, C₁-C₃ alkyl groups, C₁-C₃ alkoxy groups, C₁-C₃ halo alkyl groups, or C₁-C₃ halo alkoxy groups.

R⁶ is comprised of a C₁-C₈ alkyl group. R⁷ is comprised of a C₁-C₈ alkyl, allyl group, a morpholine, a piperazine, an N-substituted piperazine with R⁵, or a 5- or 6-membered heterocycle containing N, O, S or any combination thereof.

In another embodiment, the compounds of the present invention may be comprised of either an oxygen or sulfur in the X position defined in formula I. To this end, these embodiments may be defined by formula IIa and IIb below:

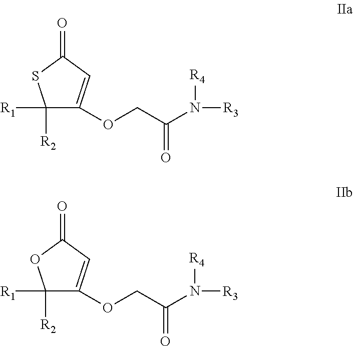

wherein each of R¹-R⁴ are defined within the embodiments discussed above.

In another embodiment, R³ is comprised of a hydrogen. R⁴ is comprised of an aryl group which may be optionally substituted with R⁸ and/or R⁸ as set forth in formula III below:

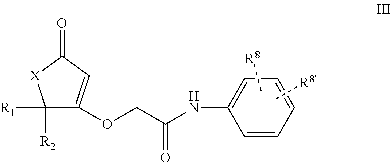

wherein each of R¹-R² are defined within the embodiments discussed above. R⁸ and R^{8′} are independently either absent from the structure or comprised of a halogen atom, a C₁-C₃ alkyl group, a C₁-C₃ haloalkyl group, —OR⁵—SR⁵—CN, —CONH₂, —SO₂NH₂, —C(O)OR⁶—CONHR⁷ or a 5- or 6-membered cycloalkyl or heterocyclic ring. The latter 5- or 6-membered cycloalkyl or heterocyclic ring is optionally aromatic, optionally fused to two adjacent carbon atoms of the aryl ring in the R⁴ position and/or is optionally substituted with R⁵. R⁵, R⁶, and R⁷ are any of the embodiments defined herein.

In a further embodiment of formula III, X may be comprised of an S or O as follows:

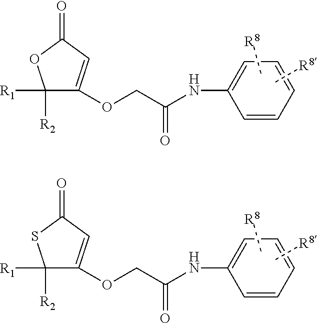

wherein R¹-R², R⁸ and R^{8′} are as defined herein.

In a further embodiment, R³ and R⁴ along with the atoms and bonds to which they are attached, form a 5-7 membered ring having at least one nitrogen atom within the ring structure. In certain embodiments the 5-7 membered ring may have at least two nitrogen atoms. In even further embodiments, R³ and R⁴ along with the atoms and bonds to which they are attached, form a 6-membered ring having two nitrogen atoms in a para position with respect to each other. In any of the foregoing embodiments the heterocyclic ring structure may be optionally substituted with R⁵ or any other substitution group discussed herein. To this end, embodiments of the foregoing may be represented by the structures of formula IV below:

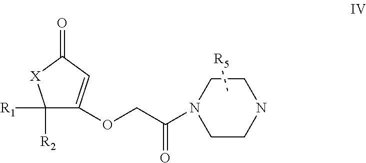

wherein R¹, R², and R⁵ are any of the embodiments defined above.

In a further embodiment of formula IV, X may be comprised of an S or O as follows:

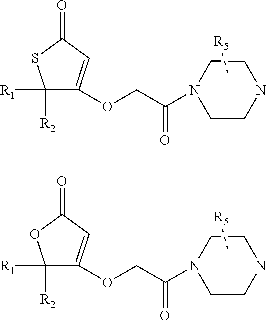

wherein R¹, R², and R⁵ are any of the embodiments defined above.

In certain non-limiting embodiments of the present invention R¹ is comprised of a straight or branched chain C₆-C₈ alkyl group. In further non-limiting embodiments, R¹ is comprised of a straight or branched chain C₈ alkyl group. In even further non-limiting embodiments, R¹ may be represented by the formula —(CH₂)₇CH₃.

In certain non-limiting embodiments of the present invention R² is comprised of a straight or branched chain C₁-C₃ alkyl group. In even further non-limiting embodiments, R² is comprised of a methyl group.

Based on the foregoing, the structures of formulas I, II, III, and IV may be adapted as follows:

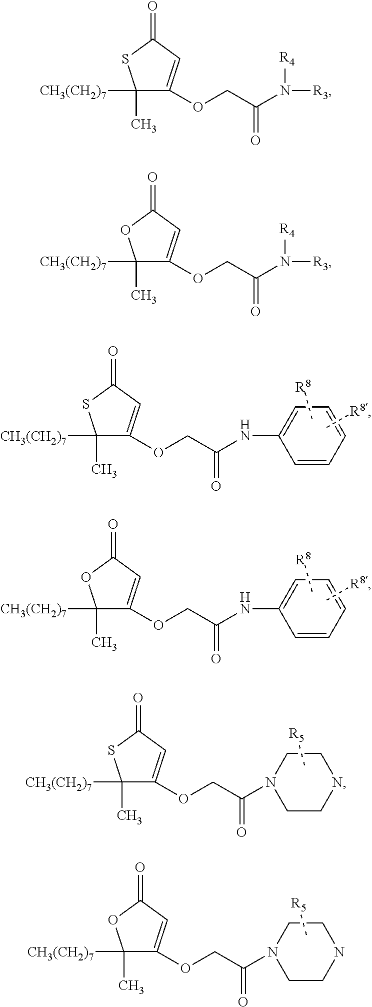

In certain embodiments the compound of the instant invention may be comprised of a compound having the following structure (referred to hereinafter as “C31”):

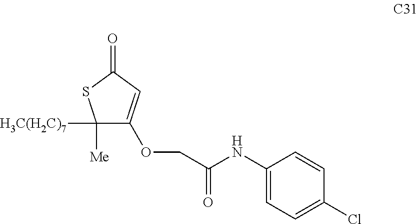

In certain embodiments the compound of the instant invention may be comprised of a compound having the following structure (referred to hereinafter as “C157”):

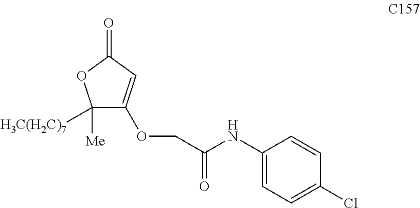

In certain embodiments the compound of the instant invention may be comprised of a compound having the following structure (referred to hereinafter as “C144”):

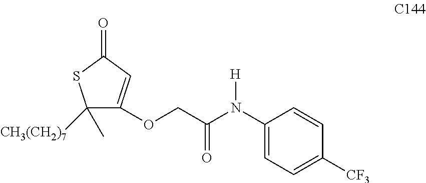

In certain embodiments the compound of the instant invention may be comprised of a compound having the following structure (referred to hereinafter as “C145”):

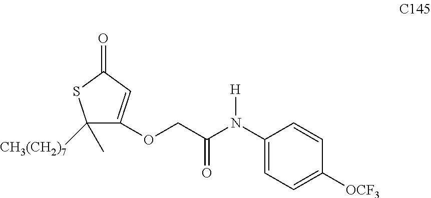

In certain embodiments the compounds of the instant invention may be comprised of a compound having the following structures (respectively referred to hereinafter as “C193”, “C138”, “C139”, “C141”, “C142”, “C178”, and “C181”):

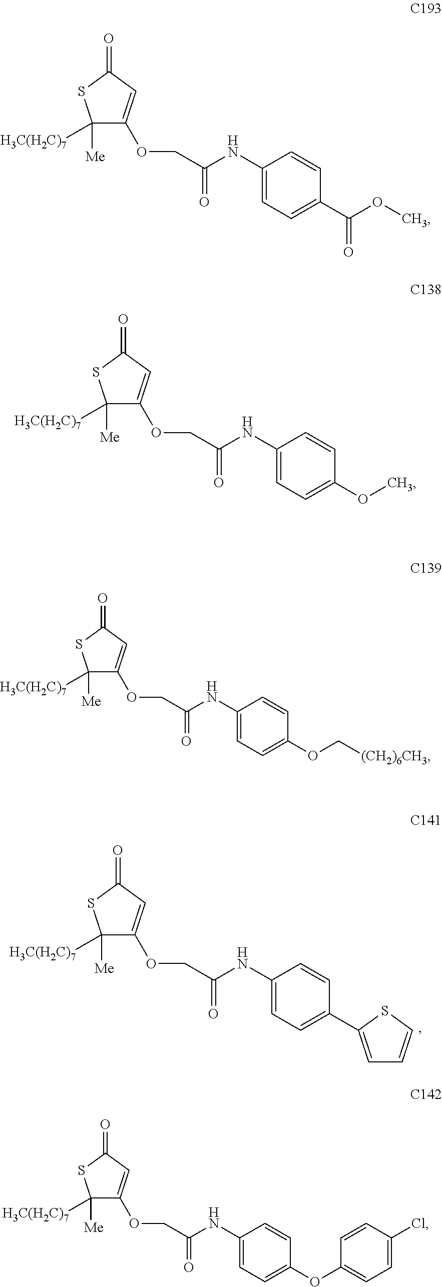

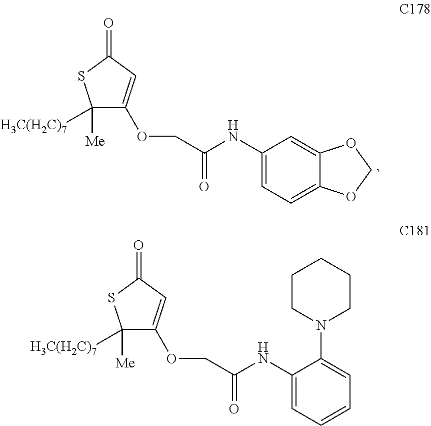

In certain embodiments the compounds of the instant invention may be any one of the following compounds:

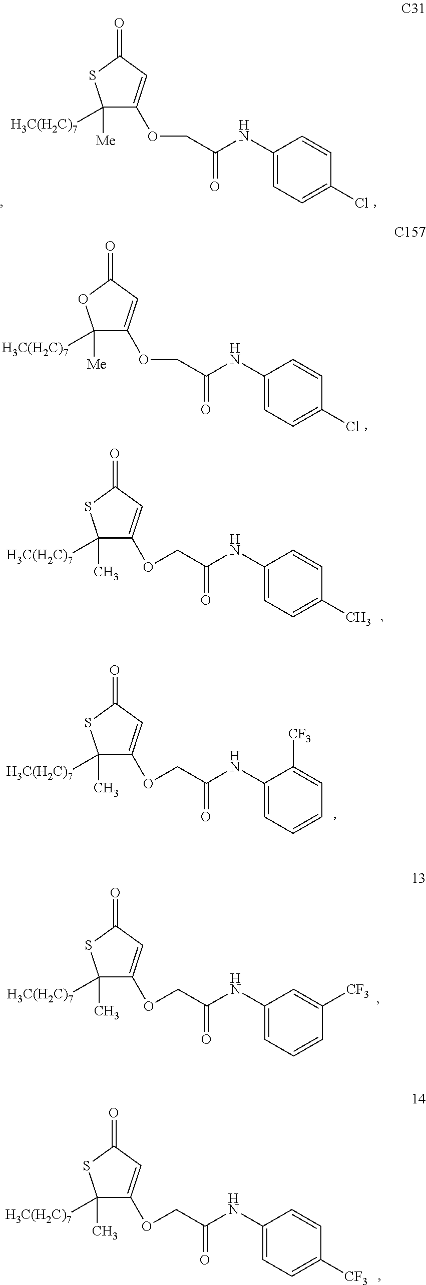

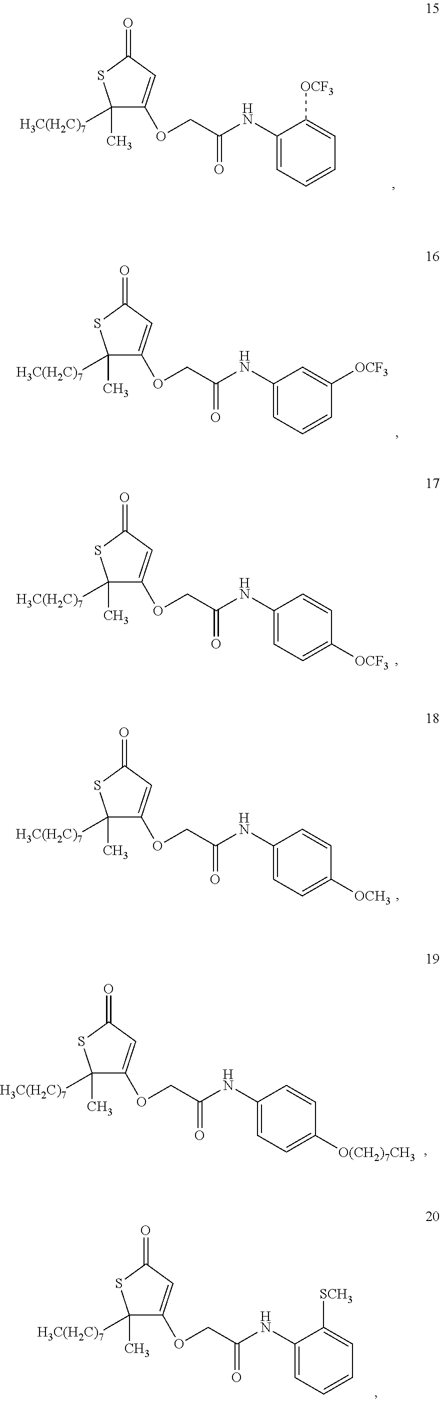

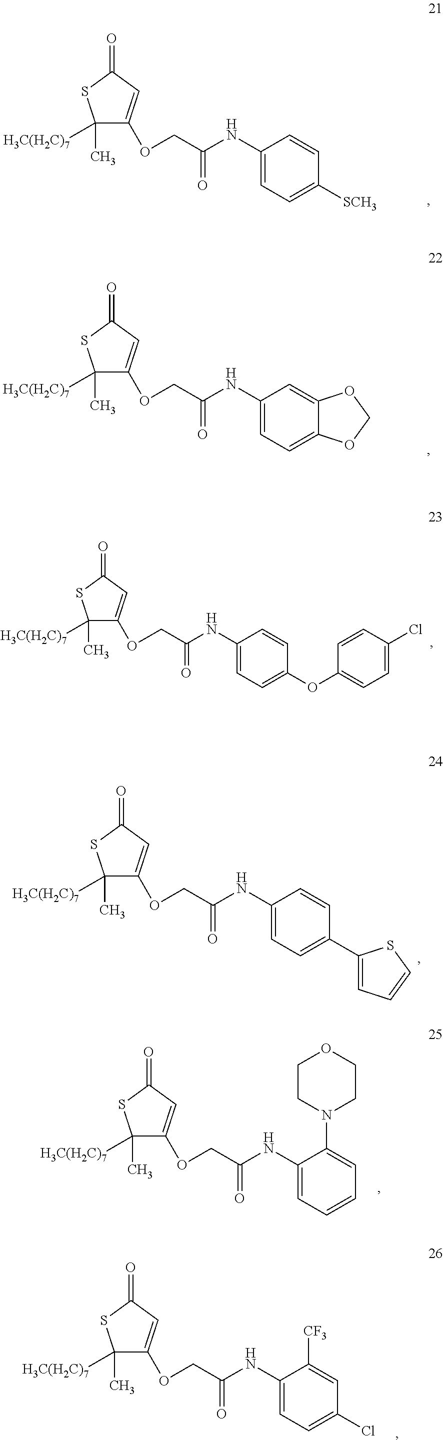

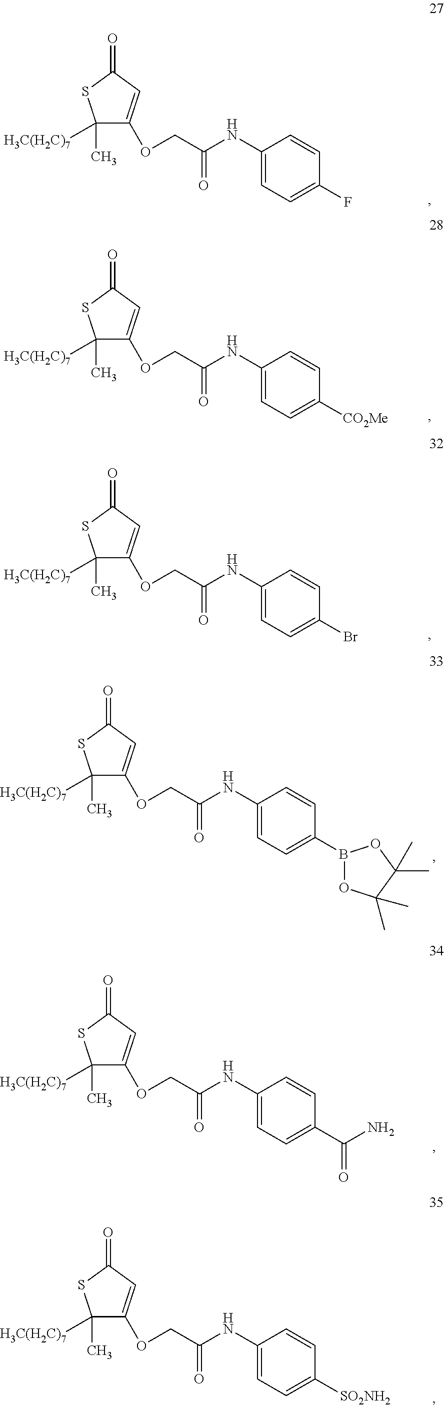

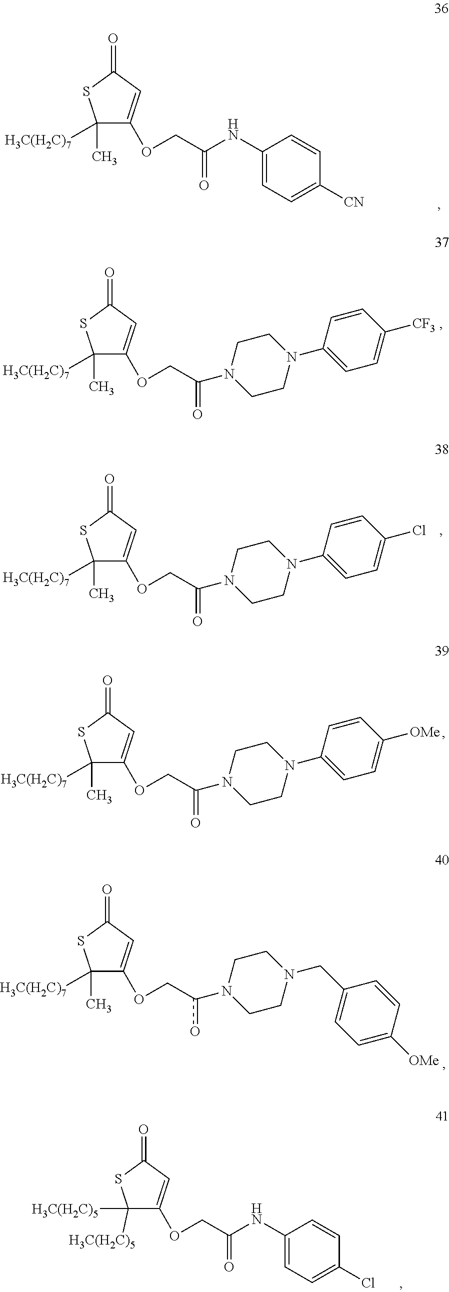

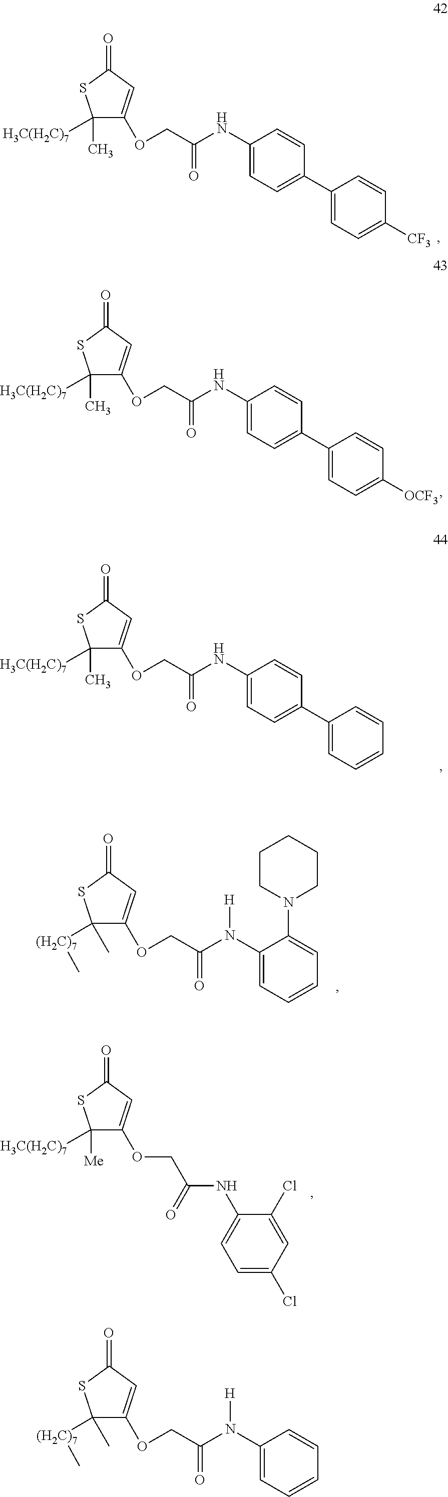

Without seeking to limit the possible scope of use of the foregoing compounds, the clinical therapeutic indications envisioned include, but are not limited to, treatment of cancers of various types, including cancers arising in many tissues whose cells over-express fatty acid synthase. One or more small molecules, or pharmaceutical salts thereof, of the present invention may be synthesized and administered as a composition used to treat and/or prevent obesity by targeted FAS activity and inhibiting fatty acid synthesis. Finally, the compound or compounds of the present invention may be synthesized and administered as a composition used to treat microbial infections due to invasive organisms which express the FAS protein, or a homolog thereof. Such microbes include, but are not limited, staphylococci and enterococci. Compounds of the present invention may be synthesized using methods known in the art or as otherwise specified herein.

Unless otherwise specified, a reference to a particular compound of the present invention includes all isomeric forms of the compound, to include all diastereomers, tautomers, enantiomers, racemic and/or other mixtures thereof. Unless otherwise specified, a reference to a particular compound also includes ionic, salt, solvate (e.g., hydrate), protected forms, and prodrugs thereof. To this end, it may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, the contents of which are incorporated herein by reference.

Based on the foregoing, one or more compounds of the present invention, either alone or in combination with another active ingredient, may be synthesized and administered as a therapeutic composition. The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. To this end, the pharmaceutical compositions may be formulated to suit a selected route of administration, and may contain ingredients specific to the route of administration. Routes of administration of such pharmaceutical compositions are usually split into five general groups: inhaled, oral, transdermal, parenteral and suppository. In one embodiment, the pharmaceutical compositions of the present invention may be suited for parenteral administration by way of injection such as intravenous, intradermal, intramuscular, intrathecal, or subcutaneous injection. Alternatively, the composition of the present invention may be formulated for oral administration as provided herein or otherwise known in the art.

As used in this specification, the terms “pharmaceutical diluent” and “pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar or another sweetener, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

Dose and duration of therapy will depend on a variety of factors, including (1) the patient's age, body weight, and organ function (M., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, the dose will be chosen to achieve serum levels of 1 ng/ml to 100 ng/ml with the goal of attaining effective concentrations at the target site of approximately 1 gg/ml to 10 μg/ml. Using factors such as this, a therapeutically effective amount may be administered so as to ameliorate the targeted symptoms of and/or treat or prevent the cancerous cells, obesity, or invasive microbial infection or diseases related thereto. Determination of a therapeutically effective amount is well within the capabilities of those skilled in the art, especially in light of the detailed disclosure and examples provided herein.

### EXAMPLES

### Example 1

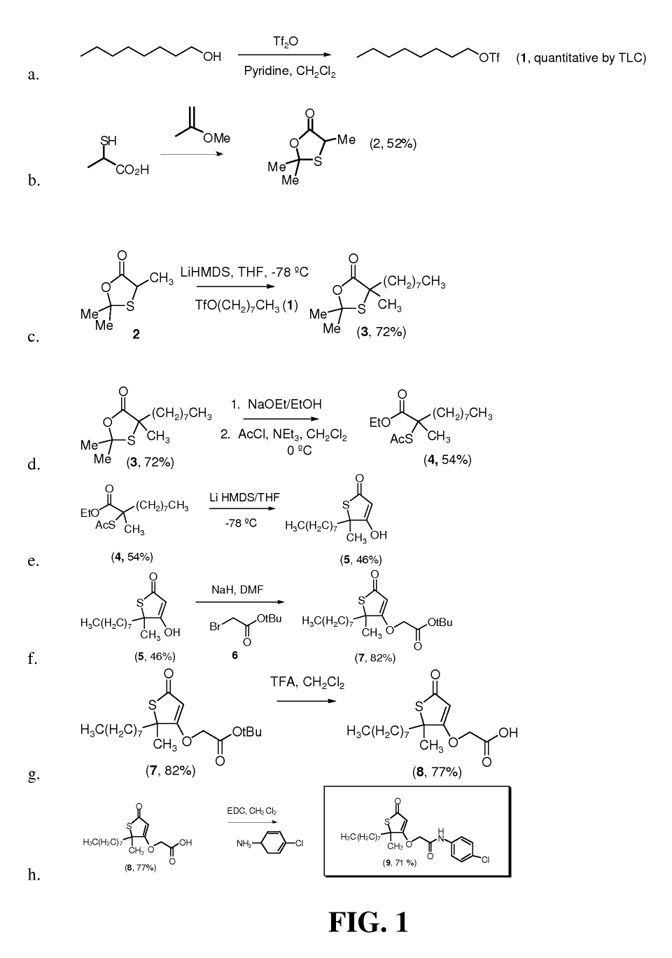

### Synthesis of C31 as Illustrated in FIG. 1

Step A—Octyl triflate (1). To octanol (4.6 g, 35.3 mmol) in CH₂Cl₂ (212 mL) cooled to −40° C. was added pyridine (freshly distilled from CaH₂, 3.28 mL, 40.6 mmol), and triflic anhydride (6.41 mL, 38.1 mmol), and the solution was allowed to stir for 20 min at −40° C. Then the reaction mixture was slowly allowed to warm up to room temperature over 3 h. The white solid was then filtered through Celite, which was washed with pentane (2×70 mL). Most of the solvents were evaporated leaving approximately 5-10 mL of solvent and a white precipitate present. Hot pentane (70 mL) was added and this mixture was filtered to remove any remaining pyridine salts. The filtrate was again evaporated to give a clear pale orange oil 1 (quantitative by TLC, rf=0.64 10% EtOAc/Hex) which was used immediately.

Step B—2,2,4-Trimethyl-[1,3]oxathiolan-5-one (2). To thiolactic acid (14.0 g, 132.0 mmol) cooled to 0° C. was added 2-methoxypropene (50.5 mL, 528 mmol) dropwise using an addition funnel. The solution was allowed to warm to room temperature, then heated to reflux for 48 h. After cooling to room temperature, Et₂O (200 mL) was added and this mixture was extracted with Na₂CO₃ (1N, 3×150 mL), and washed with brine (2×100 mL). The combined organics were dried (MgSO₄), filtered and evaporated to give a crude yellow oil, which was distilled (H₂O aspirator pressure, 25-35 torr) at 80-95° C. to give pure 2 (9.9 g, 52%). ¹H NMR (300 MHz, CDCl₃) δ 1.56 (d, J=6.9 Hz, 3H), 1.72 (s, 3H), 1.74 (s, 3H), 4.10 (q, J=6.9 Hz, 1 H). ¹³C NMR (75 MHz, CDCl₃) δ 17.9, 30.8, 31.4, 42.5, 86.2, 175.0.

Step C—2,2,5-Trimethyl-5-octyl-[1,3]-oxathiolan-4-one (3). To a mixture of LiHMDS (31.7 mL, 31.7 mmol, 1 M in THF) in THF (47 mL) at −78° C. was added 2 (4.3 g, 29.4 mmol) in THF (47 mL) dropwise by cannula, and the resulting yellow solution stirred for 30 min at −78° C. Then, octyl triflate 1 (9.0 g, 35 mmol) in pentane (8 mL) was added slowly at room temperature via cannula to the solution of the enolate at −78° C. After stifling at −78° C. for 2 h, 1 N HCl (200 mL) was added and the solution was extracted with Et₂O (3×75 mL). The combined organics were dried (MgSO₄), filtered and evaporated. Flash chromatography (2% EtOAc/hexanes) gave pure 3 (5.45 g, 72%). ¹H NMR (300 MHz, CDCl₃ δ 0.86 (bs, 3H), 1.25 (m, 10H), 1.63 (s, 3H), 1.73 (s, 3H), 1.80 (s, 3H), 1.5-1.81 (m, 4H); ¹³C NMR (75 MHz, CDCl₃) δ 14.0, 22.6, 25.5, 29.0, 29.1, 29.3, 29.4, 31.8, 32.5, 33.5, 41.4, 58.1, 84.7, 177.7.

Step D—2-Acetylsulfanyl-2-methyl-decanoic acid ethyl ester (4). To 3 (5.33 g,

20.6 mmol) in EtOH (anhydrous, 14.6 mL) was added NaOEt (2.1 M, 12.7 mL, 26.9 mmol) [freshly prepared from Na metal (1.24 g, 54 mmol) in EtOH (24 mL)] and the solution was allowed to stir at room temperature. After 30 min, the solution was poured into NH₄Cl₍ₛₐₜ₎/1 N HCl (100 mL, 3:2) and extracted with Et₂O (3×75 mL). The combined organics were then washed thoroughly with H₂O, dried (MgSO₄), filtered, evaporated and redissolved in CH₂Cl₂ (129 mL). To this precooled solution (0° C.) was added NEt₃ (4.3 mL, 30.9 mmol) and acetyl chloride (3.2 mL, 41.2 mmol). After 40 min at 0° C., NH₄Cl₍ₛₐₜ₎ (200 mL) was added and the solution was extracted with CH₂Cl₂ (3×70 mL) The combined organics were dried (MgSO₄), filtered and evaporated. Flash chromatography (5% EtOAc/hexanes) gave pure 4 (3.1 g, 54%). ¹H NMR (300 MHz, CDCl₃) δ 0.87 (t, J=6.9 Hz, 3H), 1.22-1.27 (m, 15H), 1.61 (s, 3H), 1.75-1.84 (m, 2H), 2.26 (s, 3H), 4.18 (q, J=7.1 Hz, 2H); ¹³C NMR (75 MHz, CDCl₃). δ 13.9, 14.1, 22.6, 23.4, 24.4, 29.1, 29.2, 29.6, 30.3, 31.8, 38.3, 55.8, 61.5, 173.1, 195.8. IR (NaCl) 3430, 1868, 1693, 1644 cm⁻¹; Anal. (C₁₅H₂₈O₃S) C, H.

Step E—4-Hydroxy-5-methyl-5-octyl-5-H-thiophen-2-one (5). To 4 (3.11 g, 10.8 mmol) in THF (155 mL) at −78° C. was added LiHMDS (13.4 mL, 13.4 mmol, 1.0 M in THF) and the solution was allowed to slowly warm over a 2 h period to −5° C. and then kept at −5° C. for an additional 20 min. The solution was then poured into 1 N HCl (200 mL) and extracted with Et₂O (3×100 mL). The combined organics were dried (MgSO₄), filtered and evaporated. Flash chromatography (20% EtOAc/2% CH₃CO₂H/Hexanes) gave 5 (1.2 g, 46%). ¹H NMR (300 MHz, CDCl₃) (keto-tautomer) δ 0.86 (t, J=6.7 Hz, 3H), 1.19-1.24 (m, 10H), 1.48-1.53 (m, 2H), 1.65 (s, 3H), 1.77-1.85 (m, 1H), 1.94-2.01 (m, 1H), 3.36 (s, 2H); ¹H NMR (300 MHz, MeOD) (enol tautomer) 0.87-0.89 (m, 3H), 1.29 (m, 10H), 3.29 (s, 3H), 1.81-1.87 (m, 2 H); ¹³C NMR (75 MHz, MeOD) (enol tautomer) δ14.7, 23.8, 26.4, 27.1, 30.5, 30.6, 30.8, 33.2, 39.8, 61.3, 103.1 (m), 189.8, 197.8. IR (NaCl) 3422, 1593 cm⁻¹; Anal. (C₁₃H₂₂O₂S), C, H.

Step F—5-Methyl-5-octyl-2-oxo-thiophen-4-yloxy)-acetic acid tert-butyl ester (7). To 5 (1.4 g, 5.8 mmol) in DMF (23 mL) cooled to −40° C. was added NaH (326 mg, 8.15 mmol, 60% in mineral oil) and the solution was allowed to warm and stir at 0° C. for 30 min. t-Butyl bromoacetate 6 (1.29 mL, 8.73 mmol) was then added directly and the mixture was allowed to warm and stir for 3 h at room temperature. NH₄Cl₍ₛₐₜ₎/1 N HCl (6:1, 100 mL) was added and the solution was extracted with Et₂O (3×70 mL). The combined organics were washed with H₂O, dried (MgSO₄), filtered and evaporated. Flash chromatography (15% EtOAc/hexanes) gave pure 7 (1.7 g, 82%). ¹H NMR (300 MHz, CDCl₃) δ 0.86 (t, J=6.9 Hz, 3 H), 1.24 (s, 12H), 1.49 (s, 9H), 1.68 (s, 3H), 1.83-1.86 (m, 2H), 4.43 (s, 2H), 5.19 (s, 1H); ¹³C NMR (75 MHz, CDCl₃) δ 14.0, 22.6, 25.2, 26.3, 28.1, 29.2, 29.3, 29.5, 31.8, 38.9, 59.7, 68.5, 83.4, 102.1, 165.2, 185.5, 193.4. Anal. (C₁₉H₃₂O₄S) C, H.

Step G—5-Methyl-5-octyl-2-oxo-thiophen-4-yloxy)-acetic acid (8). To 7 (1.7 g, 4.7 mmol) dissolved in CH₂Cl₂ (32 mL) was added trifluoroacetic acid (TFA) (9.1 mL) and the solution was stirred at room temperature for 4-5 h. The solvents were evaporated and the crude material was chromatographed (40% EtOAc/2% CH₃CO₂H/hexanes) to give pure 8 (1.1, 77%). ¹H NMR (300 MHz, CDCl₃) δ 0.86 (t, J=6.9 Hz, 3H), 1.24 (s, 11H), 1.47-1.48 (m, 1H), 1.68 (s, 3H), 1.84-1.88 (m, 2H), 4.62 (s, 2H), 5.31 (s, 1H); ¹³C NMR (75 MHz, CDCl₃) δ 14.1, 22.6, 25.1, 26.1, 29.2, 29.3, 29.5, 31.8, 38.9, 60.1, 67.7, 102.4, 169.8, 185.8, 195.4. IR (NaCl) 3442, 1645 cm⁻¹; Anal. (C₁₅H₂₄O₄S) C, H.

Step H—N-(4-Chlorophenyl)-(5-Methyl-5-octyl-2-oxo-thiophen-4-yloxy)-acetamide (9). To a cooled solution of 8 (1.165 g, 3.9 mmol, 1.0 equiv.) in CH₂Cl₂ at 0° C. was added EDC (1.196 g, 6.24 mmol, 1.6 equiv.), DMAP (71.3 mg, 0.58 mmol, 0.15 equiv.) and 4-Chloroaniline (697 mg, 5.46 mmol, 1.4 equiv.) and the solution were allowed to stir at 0° C. for 1 h. The reaction was slowly allowed to warm to room temperature and stir for 12 h. The mixture was poured into saturated aq. NH₄Cl:1 N HCl (4:1) and extracted with CH₂Cl₂. The organics were combined, dried (MgSO4), filtered and evaporated. Flash chromatography 30% EtOAc-40% EtOAc/hexane gave pure compound (1.132 g, 71% yield) as a white powder. The compound was then recrystallized using Ether:Chloroform (9:1) to give white crystalline solid. ¹H NMR (300 MHz, CDCl₃) δ 0.83 (t, J=7.2 Hz, 3H), 1.21 (m, 11H), 1.45-1.51 (m, 1H), 1.72 (s, 3H), 1.85-1.89 (m, 2H), 4.53 (s, 2H), 5.38 (s, 1H), 7.30 (d, J=8.8 Hz, 2H), 7.45 (d, J=8.8 Hz, 2 H), 7.85 (bs, 1H); ¹³C NMR (100 MHz, CDCl₃) δ 14.1, 22.6, 25.3, 26.4, 29.2, 29.3, 29.5, 31.8, 39.0, 59.4, 70.2, 103.6, 121.3, 129.3, 130.5, 134.9, 163.4, 183.8 and 193.0.

### Example 2

### Synthesis of C157

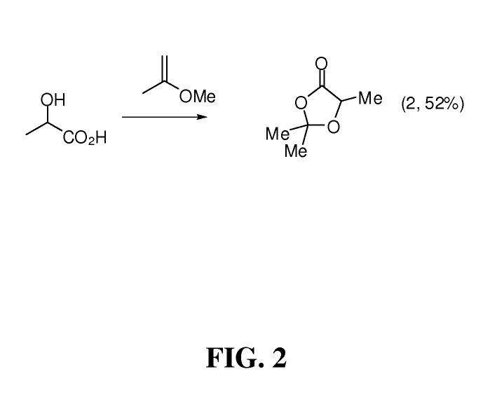

To make C157, the same process as was used to make C31 can be employed, as illustrated in FIG. 1, except that in the second step, lactic acid is used instead of thiolactic acid, as shown in FIG. 2.

### Example 3

### General Procedure for Purification of Compounds

To a cooled solution (0° C.) of 8 (0.2 mmol, 1.0 equiv.) in CH₂Cl₂ (3.0 mL) was added 1-[3-(dimethylamino)propyl]-3-ethylcarbodiimide hydrochloride (EDC) (0.32 mmol, 1.6 equiv.), aniline derivative (0.22 mmol, 1.1 equiv.), and DMAP (0.03 mmol, 0.15 equiv). The mixture was stirred at 0° C. for 30 min, then warmed to room temperature and stirred for 4 h. The solution was poured into saturated aqueous NH₄Cl (10 ml) and extracted with CH₂Cl₂ (3×10 ml). The combined organics were dried (MgSO₄), filtered and evaporated to give crude product. Flash chromatography with 30% EtOAc/Hex gave pure product.

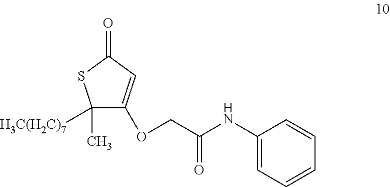

2-(2-Methyl-2-octyl-5-oxo-2,5-dihydro-thiophen-3-yloxy)-N-phenyl-acetamide (10). To 8 (45.0 mg, 0.15 mmol) and aniline (17.0 L, 0.18 mmol), following general procedure A compound 10 was obtained (50.0 mg, 67%) as an oil. ¹H NMR (400 MHz, CDCl₃) δ 0.86 (t, J=8.0 Hz, 3H), 1.17-1.35 (m, 11H), 1.50-1.60 (m, 1H), 1.75 (s, 3H), 1.87-1.93 (m, 2H), 4.56 (s, 2H), 5.41 (s, 1H), 7.18 (t, J=8.0 Hz, 1H), 7.37 (t, J=8.0 Hz, 2H), 7.52 (d, J=8.0 Hz, 2H), 8.11 (s, 1H); ¹³C NMR (100 MHz, CDCl₃) δ14.0, 22.6, 25.3, 26.4, 29.2, 29.3, 29.5, 31.8, 39.0, 59.4, 70.3, 103.4, 120.2, 125.4, 129.2, 136.3, 163.4, 183.9, 193.0.

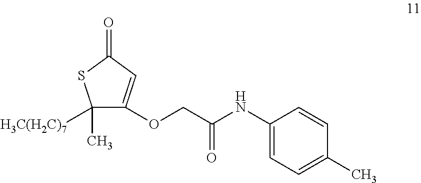

2-(2-Methyl-2-octyl-5-oxo-2,5-dihydro-thiophen-3-yloxy)-N-p-tolyl-acetamide (11). To 8 (45.0 mg, 0.15 mmol) and 4-methyl aniline (19.2 mg, 0.18 mmol), following general procedure A compound II was obtained (51.0 mg, 65%) as a solid. ¹H NMR (400 MHz, CDCl₃) δ 0.86 (t, J=8.0 Hz, 3H), 1.15-1.35 (m, 11H), 1.49-1.60 (m, 1H), 1.74 (s, 3H), 1.87-1.93 (m, 2H), 2.33 (s, 3H), 4.54 (s, 2H), 5.39 (s, 1H), 7.15 (d, J=8.0 Hz, 2H), 7.39 (d, J=8.0 Hz, 2 H), 7.92 (s, 1H); ¹³C NMR (75 MHz, CDCl₃) δ 14.0, 20.9, 22.6, 25.3, 26.4, 29.2, 29.3, 29.5, 31.7, 39.0, 59.4, 70.3, 103.3, 120.3, 129.7, 133.7, 135.1, 163.3, 184.0, 193.2. m.pt: 96° C.

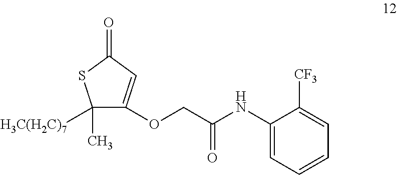

N-(2-Trifluoromethyl-phenyl)-2-(2-methyl-2-octyl-5-oxo-2,5-dihydro-thiophen-3-yloxy)-acetamide (12). To 8 (45.0 mg, 0.15 mmol) and 2-trifluoromethyl aniline (21.0 μL, 0.16 mmol), following general procedure A compound 12 was obtained (30.0 mg, 45%). ¹H NMR (500 MHz, CDCl₃) δ 0.83 (t, J=6.5 Hz, 3H), 1.14-1.25 (m, 11H), 1.51-1.56 (m, 1H), 1.72 (s, 3H), 1.89 (t, J=7.5 Hz, 2H), 4.55 (s, 2H), 5.41 (s, 1H), 7.28 (t, J=8.0 Hz, 1H), 7.60 (t, J=8.0 Hz, 1H), 7.65 (d, J=8.0 Hz, 1H), 8.37 (d, J=8.0 Hz, 1H), 8.48 (s, 1H).

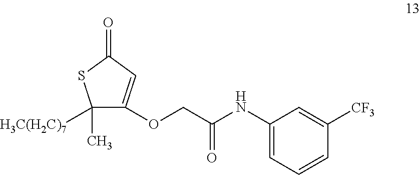

N-(3-Trifluoromethyl-phenyl)-2-(2-methyl-2-octyl-5-oxo-2,5-dihydro-thiophen-3-yloxy)-acetamide (13). To 8 (45.0 mg, 0.15 mmol) and 3-trifluoromethyl aniline (21.0 μL, 0.16 mmol), following general procedure A compound 13 was obtained (54.3 mg, 82%). ¹H NMR (500 MHz, CDCl₃) δ 0.84 (t, J=6.0 Hz, 3H), 1.14-1.30 (m, 11H), 1.55-1.59 (m, 1H), 1.75 (s, 3H), 1.91 (m, 2H), 4.58 (s, 2H), 5.42 (s, 1H), 7.43 (d, J=8.0 Hz, 1H), 7.48 (t, J=8.0 Hz, 1H), 7.74 (d, J=8.0 Hz, 1H), 7.78 (s, 1H), 7.94 (s, 1H).

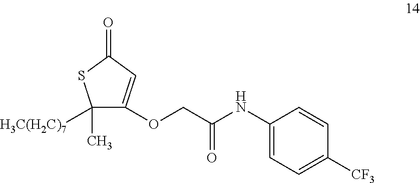

2-(2-Methyl-2-octyl-5-oxo-2,5-dihydro-thiophen-3-yloxy)-N-(4-trifluoromethyl-phenyl)-acetamide (14). To 8 (60.0 mg, 0.2 mmol) and 4-trifluoromethyl aniline (30.0 μL, 0.24 mmol), following general procedure A compound 14 was obtained (48.0 mg, 54%) as a solid. ¹H NMR (300 MHz, CDCl₃) δ 0.86 (t, J=6.0 Hz, 3H), 1.17-1.33 (m, 11H), 1.48-1.60 (m, 1H), 1.76 (s, 3H), 1.90-1.98 (m, 2H), 4.61 (s, 2H), 5.43 (s, 1H), 7.61 (d, J=9.0 Hz, 2H), 7.67 (d, J=9.0 Hz, 2H), 8.18 (s, 1H); ¹³C NMR (75 MHz, CDCl₃) δ 14.0, 22.6, 25.3, 26.4, 29.2, 29.3, 29.5, 31.8, 39.0, 59.6, 70.3, 103.4, 119.7, 126.4, 126.5, 126.8, 139.5, 163.7, 184.2, 193.5. m.pt: 87° C.

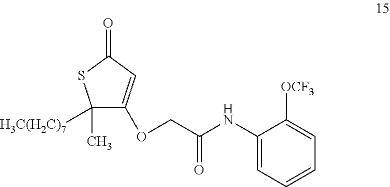

N-(2-Trifluoromethoxy-phenyl)-2-(2-methyl-2-octyl-5-oxo-2,5-dihydro-thiophen-3-yloxy)-acetamide (15). To 8 (45.0 mg, 0.15 mmol) and 2-trifluoromethoxy aniline (23.0 μL, 0.17 mmol), following general procedure A compound 15 was obtained (40.0 mg, 58%). ¹H NMR (500 MHz, CDCl₃) δ 0.83 (t, J=5.5 Hz, 3H), 1.17-1.31 (m, 11H), 1.49-1.58 (m, 1H), 1.73 (s, 3H), 1.89 (m, 2H), 4.55 (s, 2H), 5.41 (s, 1H), 7.17 (t, J=8.0 Hz, 1H), 7.31 (m, 2H), 8.40 (s, 1H), 8.48 (d, J=9.0 Hz, 1H).

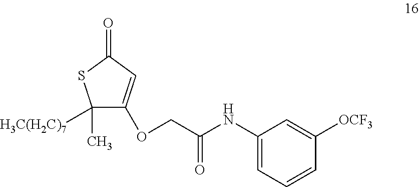

N-(3-Trifluoromethoxy-phenyl)-2-(2-methyl-2-octyl-5-oxo-2,5-dihydro-thiophen-3-yloxy)-acetamide (16). To 8 (45.0 mg, 0.15 mmol) and 3-trifluoromethoxy aniline (22.0 μL, 0.17 mmol), following general procedure A compound 16 was obtained (54.4 mg, 79%). ¹H NMR (500 MHz, CDCl₃) δ0.84 (t, J=6.5 Hz, 3H), 1.17-1.31 (m, 11H), 1.49-1.58 (m, 1 H), 1.74 (s, 3H), 1.90 (m, 2H), 4.57 (s, 2H), 5.41 (s, 1H), 7.04 (m, 1H), 7.37 (m, 2H), 7.55 (s, 1H), 7.92 (s, 1H).

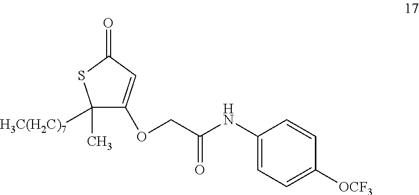

2-(2-Methyl-2-octyl-5-oxo-2,5-dihydro-thiophen-3-yloxy)-N-(4-trifluoromethoxy-phenyl)-acetamide (17). To 8 (60.0 mg, 0.2 mmol) and 4-trifluoromethoxy aniline (29.5 μL, 0.24 mmol), following general procedure A compound 17 was obtained (62.0 mg, 68%) as a solid. ¹H NMR (300 MHz, CDCl₃) δ 0.86 (t, J=6.0 Hz, 3H), 1.13-1.27 (m, 11 H), 1.47-1.56 (m, 1H), 1.75 (s, 3H), 1.88-1.96 (m, 2H), 4.59 (s, 2H), 5.42 (s, 1H), 7.20 (dt, J=3.0, 9.0 Hz, 2H), 7.57 (dt, J=3.0, 9.0 Hz, 2H), 8.11 (s, 1H); ¹³C NMR (75 MHz, CDCl₃) 814.0, 22.6, 25.3, 26.3, 29.2, 29.3, 29.5, 31.8, 39.0, 59.6, 70.3, 103.4, 118.7, 121.4, 121.9, 135.0, 146.0, 163.5, 184.3, 193.5. m.pt: 87° C.

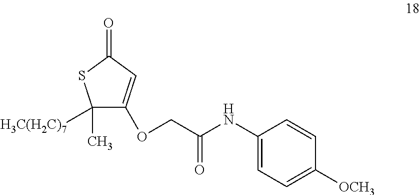

N-(4-Methoxy-phenyl)-2-(2-methyl-2-octyl-5-oxo-2,5-dihydro-thiophen-3-yloxy)-acetamide (18). To 8 (60.0 mg, 0.2 mmol) and 4-methoxy aniline (29.5 mg, 0.24 mmol), following general procedure A compound 18 was obtained (64.0 mg, 79%) as a solid. ¹H NMR (400 MHz, CDCl₃) δ 0.86 (t, J=8.0 Hz, 3H), 1.17-1.31 (m, 11H), 1.52-1.57 (m, 1H), 1.75 (s, 3 H), 1.87-1.93 (m, 2H), 3.80 (s, 3H), 4.55 (s, 2H), 5.41 (s, 1H), 6.89 (dt, J=3.0, 8.0 Hz, 2H), 7.41 (dt, J=3.0, 8.0 Hz, 2H), 7.79 (s, 1H); ¹³C NMR (100 MHz, CDCl₃) δ14.1, 22.6, 25.3, 26.4, 29.2, 29.3, 29.5, 31.8, 39.0, 55.5, 59.3, 70.3, 103.4, 114.3, 122.1, 129.0, 157.0, 163.2, 184.0, 193.2. m.pt. 99° C.

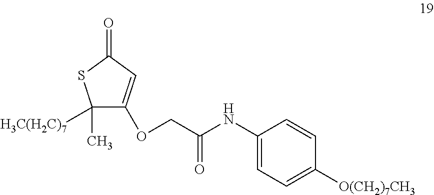

2-(2-Methyl-2-octyl-5-oxo-2,5-dihydro-thiophen-3-yloxy)-N-(4-octyloxy-phenyl)-acetamide (19). To 8 (60.0 mg, 0.2 mmol) and 4-Octyloxy aniline (53.0 mg, 0.24 mmol), following general procedure A compound 19 was obtained (76.0 mg, 75%) as a solid. ¹H NMR (400 MHz, CDCl₃) δ 0.85 (t, J=8.0 Hz, 3H), 0.88 (t, J=8.0 Hz, 3H), 1.17-1.35 (m, 19 H), 1.38-1.48 (m, 2H), 1.51-1.58 (m, 1H), 1.73-1.80 (m, 2H), 1.74 (s, 3H), 1.88-1.92 (m, 2H), 3.93 (t, J=8.0 Hz, 2H), 4.54 (s, 2H), 5.39 (s, 1H), 6.87 (dt, J=4.0, 8.0 Hz, 2H), 7.40 (dt, J=4.0, 8.0 Hz, 2H), 7.83 (s, 1H); ¹³C NMR (100 MHz, CDCl₃) δ 14.0, 22.5, 22.6, 25.3, 25.9, 26.4, 29.1, 29.2, 29.3, 29.5, 31.8, 39.0, 59.4, 68.3, 70.3, 103.4, 114.9, 122.1, 129.0, 156.8, 163.2, 183.9, 193.0. m. pt: 64° C.

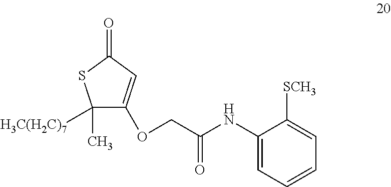

2-(2-Methyl-2-octyl-5-oxo-2,5-dihydro-thiophen-3-yloxy)-N-(2-methylsulfanyl-phenyl)-acetamide (20). To 8 (45.0 mg, 0.15 mmol) and 2-methylthio aniline (20.0 μL, 0.16 mmol), following general procedure A compound 20 was obtained (50.0 mg, 79%). ¹H NMR (500 MHz, CDCl₃) δ 0.83 (t, J=5.5 Hz, 3H), 1.17-1.33 (m, 11H), 1.49-1.58 (m, 1H), 1.78 (s, 3H), 1.91-2.01 (m, 2H), 2.38 (s, 3H), 4.56 (s, 2H), 5.42 (s, 1H), 7.13 (t, J=8.0 Hz, 1H), 7.33 (t, J=8.0 Hz, 1H), 7.52 (d, J=8.0 Hz, 1H), 8.41 (d, J=8.0 Hz, 1H), 9.35 (s, 1

H).

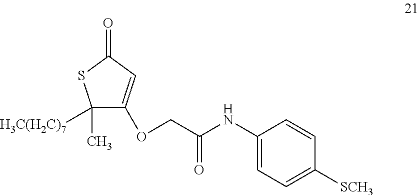

2-(2-Methyl-2-octyl-5-oxo-2,5-dihydro-thiophen-3-yloxy)-N-(4-methylsulfanyl-phenyl)-acetamide (21). To 8 (45.0 mg, 0.15 mmol) and 3-trifluoromethoxy aniline (22.0 μL, 0.17 mmol), following general procedure A compound 21 was obtained (21.0 mg, 49%). ¹H NMR (500 MHz, CDCl₃) δ 0.84 (t, J=7.0 Hz, 3H), 1.15-1.29 (m, 11H), 1.50-1.57 (m, 1H), 1.73 (s, 3H), 1.88-1.92 (m, 2H), 2.45 (s, 3H), 4.53 (s, 2H), 5.38 (s, 1H), 7.23 (d, J=8.5 Hz, 2H), 7.42 (d, J=8.5 Hz, 2H), 7.81 (s, 1H).

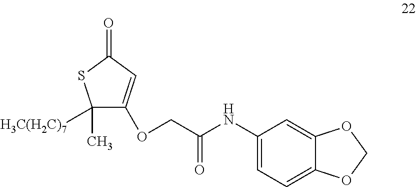

N-Benzo[1,3]dioxol-5-yl-2-(2-methyl-2-octyl-5-oxo-2,5-dihydro-thiophen-3-yloxy)-acetamide (22). To 8 (45.0 mg, 0.15 mmol) and Benzo[1,3]dioxol-5-ylamine (24.7 mg, 0.18 mmol), following general procedure A compound 22 was obtained (51.0 mg, 61%) as a solid. ¹H NMR (400 MHz, CDCl₃) δ 0.86 (t, J=8.0 Hz, 3H), 1.16-1.35 (m, 11H), 1.49-1.62 (m, 1H), 1.74 (s, 3H), 1.86-1.92 (m, 2H), 4.54 (s, 2H), 5.40 (s, 1H), 5.97 (s, 2H), 6.76 (d, J=8.0 Hz, 1H), 6.80 (dd, J=4.0, 8.0 Hz, 1H), 7.21 (d, J=4.0 Hz, 1H), 7.84 (s, 1H); ¹³C NMR (75 MHz, CDCl₃) δ 14.0, 22.6, 25.3, 26.4, 29.2, 29.3, 29.5, 31.7, 39.0, 59.4, 70.3, 101.5, 102.9, 103.4, 108.2, 113.5, 130.4, 145.1, 148.0, 163.3, 183.9, 193.2. m.pt: 102° C.

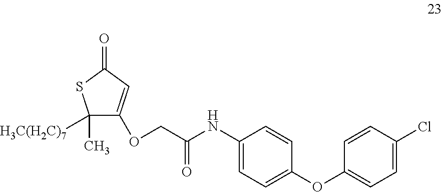

N-[4-(4-Chloro-phenoxy)-phenyl]-2-(2-methyl-2-octyl-5-oxo-2,5-dihydro-thiophen-3-yloxy)-acetamide (23). To 8 (60.0 mg, 0.2 mmol) and 4-(4-Chloro-phenoxy)-phenylamine (52.5 mg, 0.24 mmol), following general procedure A compound 23 was obtained (81.0 mg, 81%) as a solid. ¹H NMR (400 MHz, CDCl₃) δ 0.86 (t, J=6.0 Hz, 3H), 1.16-1.28 (m, 11H), 1.53-1.63 (m, 1H), 1.76 (s, 3H), 1.89-1.94 (m, 2H), 4.58 (s, 2H), 5.44 (s, 1H), 6.92 (dt, J=3.0, 9.0 Hz, 2H), 7.01 (dt, J=3.0, 9.0 Hz, 2H), 7.29 (dt, J=3.0, 9.0 Hz, 2H), 7.49 (dt, J=3.0, 9.0 Hz, 2H), 7.74 (s, 1H); m.pt: 83° C.

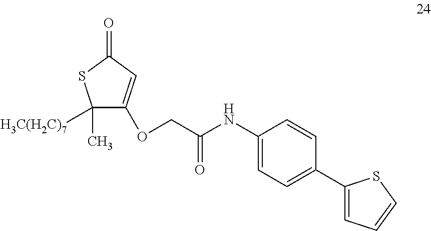

2-(2-Methyl-2-octyl-5-oxo-2,5-dihydro-thiophen-3-yloxy)-N-(4-thiophen-2-yl-phenyl)-acetamide (24). To 8 (60.0 mg, 0.2 mmol) and 4-(2-thiophenyl)-aniline (42.0 mg, 0.24 mmol), following general procedure A compound 24 was obtained (82.0 mg, 90%) as a solid. ¹H NMR (300 MHz, CDCl₃) δ 0.86 (t, J=6.0 Hz, 3H), 1.17-1.35 (m, 11H), 1.53-1.59 (m, 1H), 1.77 (s, 3H), 1.88-1.95 (m, 2H), 4.58 (s, 2H), 5.43 (s, 1H), 7.35-7.39 (m, 2H), 7.42-7.44 (m, 1H), 7.54-7.61 (m, 4H), 7.98 (s, 1H). m.pt: 130° C.

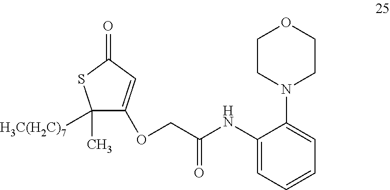

2-(2-Methyl-2-octyl-5-oxo-2,5-dihydro-thiophen-3-yloxy)-N-(2-morpholin-4-yl-phenyl)-acetamide (25). To 8 (45.0 mg, 0.15 mmol) and 2-morpholinoaniline (32.0 mg, 0.18 mmol), following general procedure A compound 25 was obtained (62.0 mg, 67%) as an oil. ¹H NMR (400 MHz, CDCl₃) δ 0.85 (t, J=8.0 Hz, 3H), 1.22-1.28 (m, 11H), 1.53-1.61 (m, 1H), 1.83 (s, 3H), 1.96-2.05 (m, 2H), 2.91 (dt, J=4.0, 10.0 Hz, 4H), 3.88 (t, J=4.0 Hz, 4H), 4.61 (s, 2H), 5.46 (s, 1H), 7.16-7.25 (m, 2H), 7.26-7.28 (m, 1H), 8.41 (dd, J=4.0, 8.0 Hz, 1H), 9.18 (s, 1H); ¹³C NMR (100 MHz, CDCl₃) δ14.0, 22.5, 25.3, 26.4, 29.1, 29.3, 29.4, 31.7, 39.2, 52.9, 59.3, 67.3, 70.8, 103.7, 120.3, 121.0, 125.1, 126.0, 132.1, 141.4, 163.4, 183.7, 192.7.

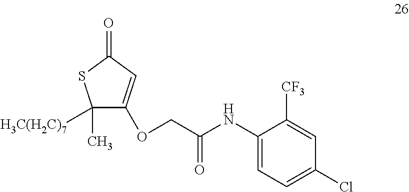

N-(4-Chloro-2-trifluoromethyl-phenyl)-2-(2-methyl-2-octyl-5-oxo-2,5-dihydro-thiophen-3-yloxy)-acetamide (26). To 8 (45.0 mg, 0.15 mmol) and 4-chloro-2-trifluoromethyl aniline (26.0 μL, 0.18 mmol), following general procedure A compound 26 was obtained (24.0 mg, 25%). ¹H NMR (300 MHz, CDCl₃) δ 0.86 (t, J=8.0 Hz, 3H), 1.14-1.25 (m, 11H), 1.51-1.56 (m, 1H), 1.74 (s, 3H), 1.86-1.92 (m, 2H), 4.57 (s, 2H), 5.43 (s, 1H), 7.58 (dd, J=4.0, 8.0 Hz, 1H), 7.65 (d, J=4.0 Hz, 1H), 8.40 (d, J=8.0 Hz, 1H), 8.48 (s, 1H).

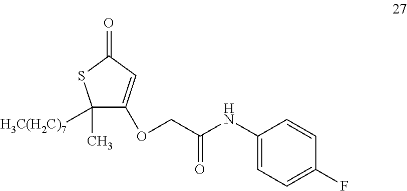

N-(4-Fluoro-phenyl)-2-(2-methyl-2-octyl-5-oxo-2,5-dihydro-thiophen-3-yloxy)-acetamide (27). To 8 (100.0 mg, 0.33 mmol) and 4-fluoroaniline (44.0 μL, 0.47 mmol), following general procedure A compound 27 was obtained (127.0 mg, 98%). ¹H NMR (400 MHz, CDCl₃) δ 0.84 (t, J=7.0 Hz, 3H), 1.23 (m, 11H), 1.48-1.55 (m, 1H), 1.73 (s, 3H), 1.87-1.91 (m, 2H), 4.55 (s, 2H), 5.39 (s, 1H), 7.03 (d, J=8.0 Hz, 2H), 7.46-7.49 (m, 2H), 8.0 (s, 1 H). ¹³C NMR (100 MHz, CDCl₃) δ 14.0, 22.6, 25.3, 26.3, 29.2, 29.3, 29.5, 31.8, 39.0, 59.5, 70.3, 103.3, 115.8, 122.1, 132.3, 159.3, 163.4, 184.2, 193.3.

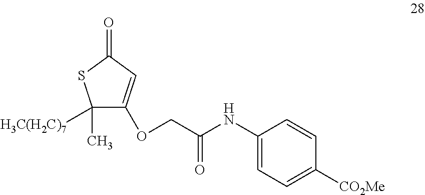

4-[2-(2-Methyl-2-octyl-5-oxo-2,5-dihydro-thiophen-3-yloxy)-acetylamino]-benzoic acid methyl ester (28). To 8 (100.0 mg, 0.33 mmol) and methyl 4-aminobenzoate (70.0 mg, 0.46 mmol), following general procedure A compound 28 was obtained (98.0 mg, 69%). ¹H NMR (400 MHz, CDCl₃) δ 0.81 (t, J=7.0 Hz, 3H), 1.22 (m, 11H), 1.49-1.52 (m, 1H), 1.72 (s, 3H), 1.87-1.91 (m, 2H), 3.87 (s, 3H), 4.59 (s, 2H), 5.38 (s, 1H), 7.61 (d, J=6.9 Hz, 2H), 7.98 (d, J=6.9 Hz, 2H), 8.5 (s, 1H). ¹³C NMR (100 MHz, CDCl₃) δ 13.9, 22.5, 25.2, 26.2, 29.1, 29.3, 29.4, 31.7, 38.9, 52.0, 59.7, 70.2, 103.1, 119.2, 126.4, 130.8, 140.8, 163.6, 166.3,184.7, 193.8.

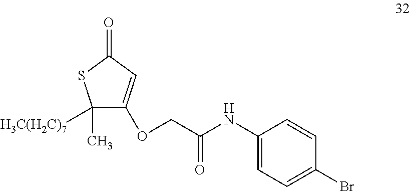

N-(4-Bromo-phenyl)-2-(2-methyl-2-octyl-5-oxo-2,5-dihydro-thiophen-3-yloxy)-acetamide (32). To 8 (300.0 mg, 1.0 mmol) and 4-bromoaniline (172 mg, 1.0 mmol), following general procedure A compound 32 was obtained (227.0 mg, 50%) as a solid. ¹H NMR (500 MHz, CDCl₃) δ 0.87 (t, J=7.0 Hz, 3H), 1.18-1.31 (m, 11H), 1.53 (m, 1H), 1.74 (s, 3H), 1.91 (t, J=8.0, 2H), 4.58 (s, 2H), 5.40 (s, 1H), 7.45 (s, 4H), 8.19 (s, 1H).

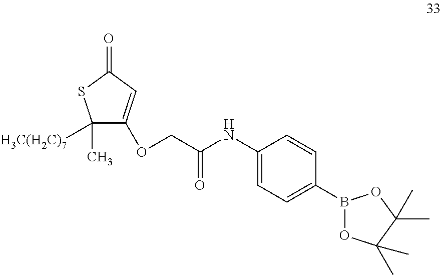

2-(2-Methyl-2-octyl-5-oxo-2,5-dihydro-thiophen-3-yloxy)-N-[4-(4,4,5,5-tetramethyl-[1,3,2]dioxaborolan-2-yl)-phenyl]-acetamide (33). To 8 (600.0 mg, 2.0 mmol) and 4-(4,4,5,5-Tetramethyl-[1,3,2]dioxaborolan-2-yl)-phenylamine (438 mg, 2.0 mmol), following general procedure A compound 33 was obtained (651.0 mg, 65%) as a solid. ¹H NMR (500 MHz, CDCl₃) δ 0.86 (t, J=8.0 Hz, 3H), 1.24-1.27 (m, 11H), 1.34 (s, 12H), 1.58 (m, 1H), 1.77 (s, 3H), 1.93 (t, J=9.0, 2H), 4.56 (s, 2H), 5.40 (s, 1H), 7.26 (s, 1H), 7.53 (d, J=8.0, 2H), 7.81 (d, J=8.0, 2H).

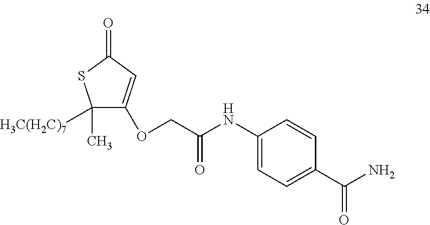

4-[2-(2-Methyl-2-octyl-5-oxo-2,5-dihydro-thiophen-3-yloxy)-acetylamino]-benzamide (34). To 8 (114.0 mg, 0.38 mmol) and 4-aminobenzamide (52 mg, 0.38 mmol), following general procedure A compound 34 was obtained (103.0 mg, 65%) as a solid. ¹H NMR (500 MHz, CD₃OD) δ 0.87 (t, J=7.0 Hz, 3H), 1.21-1.39 (m, 11H), 1.49 (s, 1H), 1.73 (s, 3H), 1.90 (m, 1H), 1.98 (d, J=13.5 Hz, 2H), 4.77 (dd, J=9.5, 15 Hz, 2H), 5.48 (s, 1H), 7.68 (d, J=9.0 Hz, 2H), 7.86 (d, J=9.0, 2H).

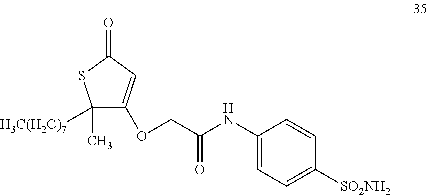

2-(2-Methyl-2-octyl-5-oxo-2,5-dihydro-thiophen-3-yloxy)-N-(4-sulfamoyl-phenyl)-acetamide (35). To 8 (105.0 mg, 0.35 mmol) and 4-Amino-benzenesulfonamide (60 mg, 0.35 mmol), following general procedure A compound 35 was obtained (37.0 mg, 24%) as a solid. ¹H NMR (500 MHz, CD₃OD) δ 0.88 (t, J=7.0 Hz, 3H), 1.28 (m, 11H), 1.48 (s, 1H), 1.73 (s, 3H), 1.91 (m, 1H), 1.98 (m, 1H), 4.78 (dd, J=7.0, 14.5 Hz, 2H), 5.47 (s, 1H), 7.75 (d, J=9.0 Hz, 2H), 7.86 (d, J=9.0, 2H).

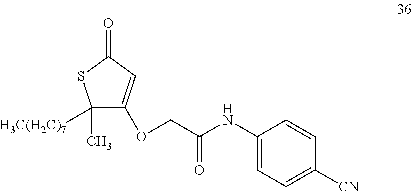

N-(4-Cyano-phenyl)-2-(2-methyl-2-octyl-5-oxo-2,5-dihydro-thiophen-3-yloxy)-acetamide (36). To 8 (107.0 mg, 0.35 mmol) and 4-Amino-benzonitrile (41 mg, 0.35 mmol), following general procedure A compound 36 was obtained (106.0 mg, 76%) as a solid. ¹H NMR (500 MHz, CDCl₃) δ 0.87 (t, J=7.0 Hz, 3H), 1.26 (m, 11H), 1.54 (s, 1H), 1.76 (s, 3

H), 1.93 (d, J=8.5 Hz, 2H), 4.64 (s, 2H), 5.42 (s, 1H), 7.64 (d, J=9.0 Hz, 2H), 7.71 (d, J=9.0 Hz, 2H), 8.43 (s, 1H).

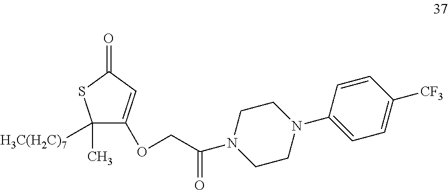

5-Methyl-5-octyl-4-[2-oxo-2-[4-(4-trifluoromethyl-phenyl)-piperazin-1-yl]-ethoxy]-5H-thiophen-2-one (37). To 8 (100.0 mg, 0.33 mmol) and 1-(4-Trifluoromethyl-phenyl)-piperazine (77 mg, 0.33 mmol), following general procedure A compound 37 was obtained (66.0 mg, 39%) as a solid. ¹H NMR (500 MHz, CDCl₃) δ 0.87 (t, J=8.0 Hz, 3H), 1.26 (m, 11H), 1.51 (s, 1H), 1.71 (s, 3H), 1.87 (m, 2H), 3.32 (s, 4H), 3.62 (s, 2H), 3.81 (s, 2H), 4.73 (s, 2H), 5.35 (s, 1H), 6.94 (d, J=9.0 Hz, 2H), 7.52 (d, J=9.0 Hz, 2H).

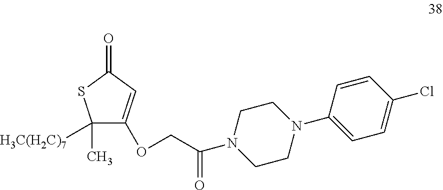

4-{2-[4-(4-Chloro-phenyl)-piperazin-1-yl]-2-oxo-ethoxy}-5-methyl-5-octyl-5H-thiophen-2-one (38). To 8 (100.0 mg, 0.33 mmol) and 1-(4-cholorphenyl)-piperazine (65 mg, 0.33 mmol), following general procedure A compound 38 was obtained (73.0 mg, 46%) as a solid. ¹H NMR (500 MHz, CDCl₃) δ 0.87 (t, J=7.0 Hz, 3H), 1.25 (m, 11H), 1.52 (s, 1H), 1.70 (s, 3H), 1.87 (m, 2H), 3.17 (s, 4H), 3.60 (s, 2H), 3.79 (s, 2H), 4.70 (s, 2H), 5.33 (s, 1 H), 6.84 (d, J=9.0 Hz, 2H), 7.24 (d, J=9.0 Hz, 2H).

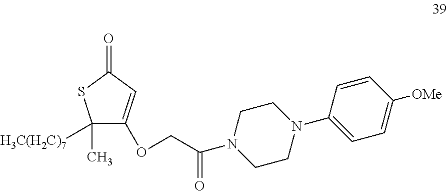

4-{2-[4-(4-Methoxy-phenyl)-piperazin-1-yl]-2-oxo-ethoxy}-5-methyl-5-octyl-5H-thiophen-2-one (39). To 8 (105.0 mg, 0.35 mmol) and 1-(4-methoxyphenyl)-piperazine (67 mg, 0.35 mmol), following general procedure A compound 39 was obtained (113.0 mg, 68%) as a solid. ¹H NMR (500 MHz, CDCl₃) δ 0.87 (t, J=6.0 Hz, 3H), 1.25 (m, 11H), 1.52 (m, 1H), 1.70 (s, 3H), 1.86 (m, 2H), 3.10 (s, 4H), 3.58 (s, 2H), 3.78 (s, 2H), 4.69 (s, 2H), 5.33 (s, 1 H), 6.85 (d, J=9.0 Hz, 2H), 6.90 (d, J=9.0 Hz, 2H).

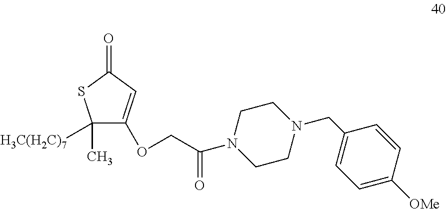

4-{2-[4-(4-Methoxy-benzyl)-piperazin-1-yl]-2-oxo-ethoxy}-5-methyl-5-octyl-5H-thiophen-2-one (40). To 8 (116.0 mg, 0.38 mmol) and 1-(4-Methoxy-benzyl)-piperazine (78 mg, 0.38 mmol), following general procedure A compound 40 was obtained (137.0 mg, 74%) as a solid. ¹H NMR (500 MHz, CDCl₃) δ 0.88 (t, J=6.0 Hz, 3H), 1.25 (m, 11H), 1.51 (m, 1 H), 1.68 (s, 3H), 1.85 (m, 2H), 2.45 (s, 4H), 3.40 (s, 2H), 3.47 (s, 2H), 3.63 (s, 2H), 3.80 (s, 3H), 4.62 (s, 2H), 5.28 (s, 1H), 6.86 (d, J=9.0 Hz, 2H), 7.21 (d, J=9.0 Hz, 2H).

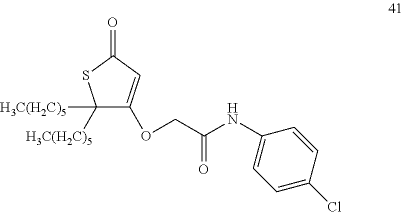

N-(4-Chloro-phenyl)-2-(2,2-dihexyl-5-oxo-2,5-dihydro-thiophen-3-yloxy)-acetamide (41). To 8 (45.0 mg, 0.16 mmol) and 2-Bromo-N-(4-chloro-phenyl)-acetamide (41 mg, 0.16 mmol), following general Procedure B, compound 41 was obtained (48.0 mg, 67.4%) as a solid. ¹H NMR (500 MHz, CDCl₃) δ 0.88 (t, J=6.0 Hz, 6H), 1.16-1.22 (m, 2H), 1.27-1.33 (m, 12H), 1.57 (s, 2H), 1.93 (m, 2H), 4.56 (s, 2H), 5.44 (s, 1H), 7.32 (d, J=9.0 Hz, 2H), 7.49 (d, J=9.0 Hz, 2H), 7.96 (s, 1H).

### Example 4

### Coupling Reaction: General Procedure

To a flame dried flask was charged with bromo compound 32 (1.0 equ.) and phenyl boronic acid (1.1 eq.), Cs₂CO₃ (1.5 eq.) and Pd(PPh₃)₄ (0.2 eq.) in DMF was heated at 100° C. for 24 h under argon. After cooling down, the reaction mixture was poured into satd. aq. Ammonium chloride solution and extracted with ether, washed with water and brine. The crude product was then subjected to column chromatography to yield the desired product

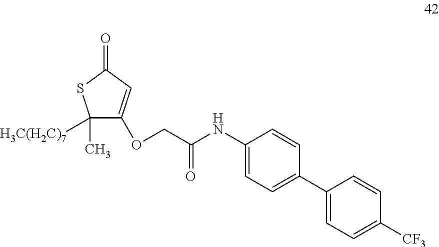

2-(2-Methyl-2-octyl-5-oxo-2,5-dihydro-thiophen-3-yloxy)-N-(4′-trifluoromethyl-biphenyl-4-yl)-acetamide (42). (KS-II-94): To 33 (130.0 mg, 0.25 mmol) and 1-Iodo-4-trifluoromethyl-benzene (46 μl, 0.31 mmol), Cs₂CO₃ (126 mg, 0.39 mmol) and Pd(PPh₃)₄ (29 mg, 0.025 mmol) following general procedure C, compound 42 was obtained (94.0 mg, 73%) as a solid. ¹H NMR (500 MHz, CDCl₃) δ 0.86 (t, J=8.0 Hz, 3H) 1.24-1.28 (m, 9H), 1.35 (m, 2H), 1.58-1.61 (m, 1H), 1.79 (s, 3H), 1.94 (m, 2H), 4.61 (s, 2H), 5.46 (s, 1H), 7.63 (d, J=6.0, 4H), 7.53 (d, J=4.5, 4H), 7.82 (s, 1H).

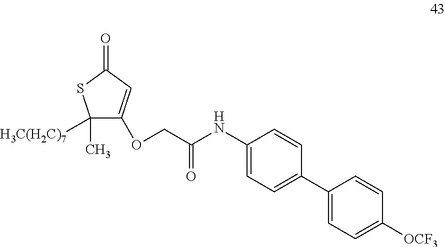

2-(2-Methyl-2-octyl-5-oxo-2,5-dihydro-thiophen-3-yloxy)-N-(4′-trifluoromethoxy-biphenyl-4-yl)-acetamide (43). (KS-II-95): To 33 (116.0 mg, 0.23 mmol) and 1-Iodo-4-trifluoromethoxy-benzene (43 μL, 0.27 mmol), Cs₂CO₃ (112 mg, 0.34 mmol) and Pd(PPh₃)₄ (26.5 mg, 0.023 mmol) following general procedure C compound 43 was obtained (80.0 mg, 65%) as a solid. ¹H NMR (500 MHz, CDCl₃) δ 0.86 (t, J=7.0 Hz, 3H) 1.26 (m, 11 H), 1.59 (m, 1H), 1.78 (s, 3H), 1.94 (t, J=8.0 Hz, 2H), 4.60 (s, 2H), 5.45 (s, 1H), 7.28 (m, 2 H), 7.61 (m, 6H), 7.85 (s, 1H).

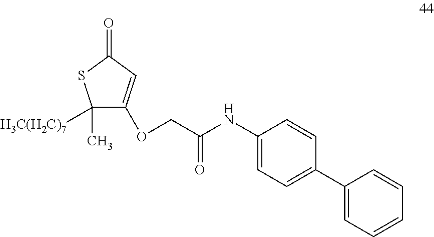

N-Biphenyl-4-yl-2-(2-methyl-2-octyl-5-oxo-2,5-dihydro-thiophen-3-yloxy)-acetamide (44). To 32 (110.0 mg, 0.24 mmol) and phenyl boronic acid (32 mg, 0.26 mmol), Cs₂CO₃ (126 mg, 0.39 mmol) and Pd(PPh₃)₄ (55.4 mg, 0.052 mmol) following general procedure C, compound 44 was obtained (44.0 mg, 41%) as a solid. ¹H NMR (500 MHz, CDCl₃) δ 0.86 (t, J=7.0 Hz, 3H) 1.22-1.34 (m, 11H), 1.57 (m, 1H), 1.77 (s, 3H), 1.93 (t, J=8.0 Hz, 2H), 4.59 (s, 2H), 5.44 (s, 1H), 7.34 (m, 1H), 7.44 (t, J=8.0 Hz, 2H), 7.57 (d, J=8.0 Hz, 2H), 7.60 (s, 4H), 7.93 (s, 1H).

### Example 5

### Process of Preparing R- and S-Enantiomers of C31

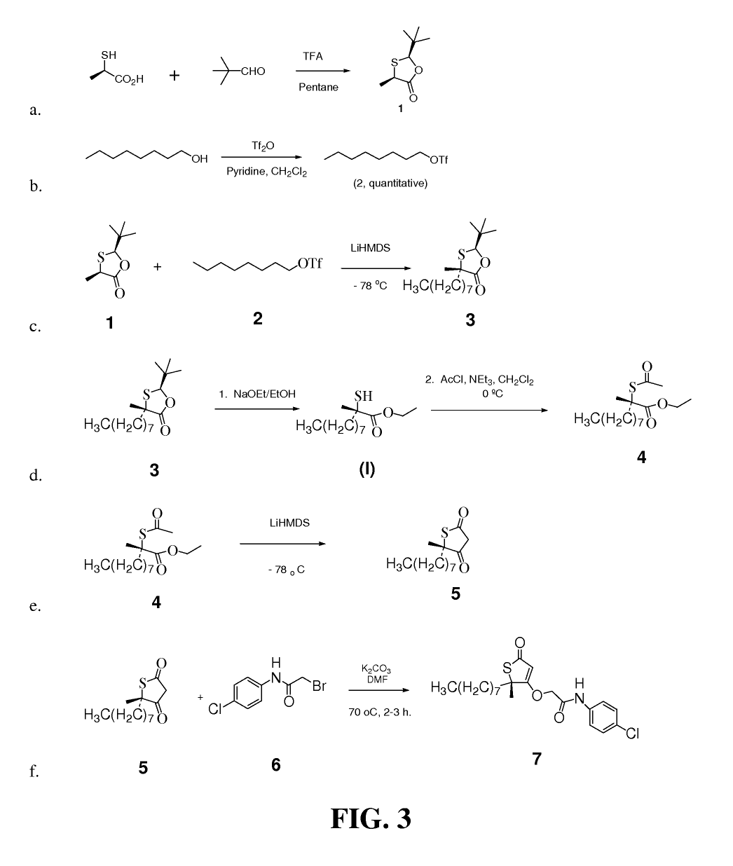

### Synthesis of S-Enantiomer—as Illustrated in FIG. 3

Step A—2-tert-Butyl-4-methyl-[1,3]oxathiolan-5-one (1). To a flame dried flask under Ar atmosphere was charged with (R)-thiolactic acid (2.5 g, 23.5 mmol), followed by pentane (20 mL) and pivaladehyde (2.82 mL, 25.9 mmol) and few drops of trifluoroacetic acid. The reaction was fitted with Dean-stark apparatus to remove the water. The solution was then heated to reflux for 48 h (55° C.) while removing the water continuously. After cooling to room temperature, the solvent was evaporated completely. The crude product was recrystallized from pentane:Ether (5:1) at −78° C. The white solid material was filtered thro crucible to give the product 1² (1.04 g, 25.4% yield). ¹H NMR (500 MHz, CDCl₃) δ 1.00 (s, 9H), 1.54 (d, J=7.0 Hz, 3H), 3.94 (q, J=6.5 Hz, 1H), 5.18 (s, 1H).

Step B—Octyl triflate (2). To octanol (4.6 g, 35.3 mmol) in CH₂Cl₂ (212 mL) cooled to −40° C. was added pyridine (freshly distilled from CaH₂, 3.28 mL, 40.6 mmol), and triflic anhydride (6.41 mL, 38.1 mmol), and the solution was allowed to stir for 20 min at −40° C. Then the reaction mixture was slowly allowed to warm up to room temperature over 3 h. The white solid was then filtered through Celite, which was washed with pentane (2×70 mL). Most of the solvents were evaporated leaving approximately 5-10 mL of solvent and a white precipitate present. Hot pentane (70 mL) was added and this mixture was filtered to remove any remaining pyridine salts. The filtrate was again evaporated to give a clear pale orange oil 2 (quantitative by TLC, rf=0.64 10% EtOAc/Hex) which was used immediately.

Step C—2-tert-Butyl-4-methyl-4-octa-1,3,5,7-tetraynyl-[1,3]oxathiolan-5-one (3). To a mixture of LiHMDS (13.8 mL, 13.8 mmol, 1 M in THF) in THF (47 mL) at −78° C. was added 1 (2.09 g, 12.0 mmol) in THF (15 mL) drop wise by cannula, and the resulting yellow solution stirred for 30 min at −78° C. Then, octyl triflate 2 (3.48 g, 13.2 mmol) in pentane (8 mL) was added slowly at room temperature via cannula to the solution of the enolate at −78° C.

After stifling at −78° C. for 2 h, 1 N HCl (200 mL) was added and the solution was extracted with Et₂O (3×75 mL). The combined organics were dried (MgSO₄), filtered and evaporated. Flash chromatography (2% EtOAc/hexanes) gave pure 3 (2.42 g, 75%). ¹H NMR (500 MHz, CDCl_{3 δ}0.86 (t, J=7.0 Hz, 3H), 0.99 (s, 9H), 1.26 (m, 10H), 1.36 (m, 1H), 1.53 (s, 4H), 1.72 (dt, J=4.0, 12.0 Hz, 1H), 1.82 (dt, J=3.5, 13.0 Hz, 1H), 5.12 (s, 1H). [α]_{D}²⁵ −40.25 (c 2.77, CHCl₃)

Step D—(S)-2-Acetylsulfanyl-2-methyl-deca-3,5,7,9-tetraynoic acid ethyl ester (4): To 3 (1.43 g, 5.0 mmol) in EtOH (anhydrous, 14.6 mL) was added NaOEt (12.5 mmol) [freshly prepared from Na metal (300 mg, 12.5 mmol) in EtOH (15 mL)] and the solution was allowed to stir at room temperature. After 30 min, the solution was poured into NH₄Cl₍ₛₐₜ₎/1 N HCl (25 mL, 3:2) and extracted with Et₂O (3×25 mL). The combined organics were then washed thoroughly with H₂O, dried (MgSO₄), filtered, evaporated to give intermediate (I), which was then redissolved in CH₂Cl₂ (25 mL). To this pre-cooled solution (0° C.) was added NEt₃ (0.83 mL, 6.0 mmol) and acetyl chloride (0.39 mL, 5.5 mmol). After 40 min at 0° C., NH₄Cl₍ₛₐₜ₎ (50 mL) was added and the solution was extracted with CH₂Cl₂ (3×20 mL). The combined organics were dried (MgSO₄), filtered and evaporated. Flash chromatography (5% EtOAc/hexanes) gave pure 4 (1.0 g, 70.6%). ¹H NMR (500 MHz, CDCl₃) δ 0.85 (t, J=7.0 Hz, 3H), 1.23-1.33 (m, 15H), 1.60 (s, 3H), 1.73-1.82 (m, 2H), 2.24 (s, 3H), 4.16 (q, J=7.0 Hz, 2 H). [α]_{D}²⁴ −7.18 (c 1.65, CHCl₃)

Step E—(S)-5-Methyl-5-octa-1,3,5,7-tetraynyl-thiophene-2,4-dione (5) (KS-II-61). To 4 (0.922 g, 3.2 mmol) in THF (15 mL) at −78° C. was added LiHMDS (4.8 mL, 4.8 mmol, 1.0 M in THF) and the solution was allowed to slowly warm over a 2 h period to −5° C. and then kept at −5° C. for an additional 20 min. The solution was then poured into 1 N HCl (20 mL) and extracted with Et₂O (3×20 mL). The combined organics were dried (MgSO₄), filtered and evaporated. Flash chromatography (20% EtOAc/2% CH₃CO₂H/Hexanes) gave 5 (0.51 g, 65.6%). ¹H NMR (500 MHz, CDCl₃) (keto-tautomer) δ0.86 (t, J=8.0 Hz, 3H), 1.26 (m, 11H), 1.49 (m, 1H), 1.63 (s, 3H), 1.80 (m, 1H), 1.94-2.01 (m, 1H), 3.34 (s, 2H); (enol tautomer characteristic peak) 5.27 (s, 1H). [α]_{D}²⁴-1.22 (c 1.44, CHCl₃)

Step F—(S)—N-(4-Chloro-phenyl)-2-(2-methyl-2-octyl-5-oxo-2,5-dihydro-thiophen-3-yloxy)-acetamide (7) (KS-II-62): A 25 mL round bottom flask was charged with 5-Methyl-5-octa-1,3,5,7-tetraynyl-thiophene-2,4-dione 5 (85.0 mg, 0.35 mmol), N-(4-chlorophenyl)-2-bromoacetamide 6 (91.0 mg, 0.36 mmol), potassium carbonate (97.0 mg, 0.7 mmol, flame dried and cooled under nitrogen atmosphere) and DMF (3.0 mL) under nitrogen atmosphere. The mixture was heated at 70° C. for 2-3 h (monitored by TLC). The solid material was filtered off and washed with diethyl ether. The solution was then diluted with ether (30 mL) and washed with water (3×15 mL), washed with saturated aqueous NH₄Cl (2×10 mL) and brine. The organic layer was dried (MgSO₄), filtered and evaporated to give crude product as a semisolid. The crude product was then recrystallized from diethyl ether:hexane (1:1) to give a white powder (basically crashed out). The product was then filtered and washed with ether:hexane (1:1). The filtrate was concentrated and recrystallized again with ether:hexane (1:1) to give white powder. The combined white powder was dried under vacuum to give the product 7 in 61.5% (88.0 g) yield. ¹H NMR (500 MHz, CDCl₃) δ 0.86 (t, J=7.0 Hz, 3H), 1.14-1.31 (m, 11H), 1.50-1.58 (m, 1H), 1.74 (s, 4H), 1.89 (m, 2H), 4.55 (s, 2H), 5.41 (s, 1H), 7.32 (d, J=9.0 Hz, 2H), 7.46 (d, J=9.0 Hz, 2H), 7.74 (s, 1H). [α]_{D}²⁵-8.29 (c 0.65, CHCl₃).

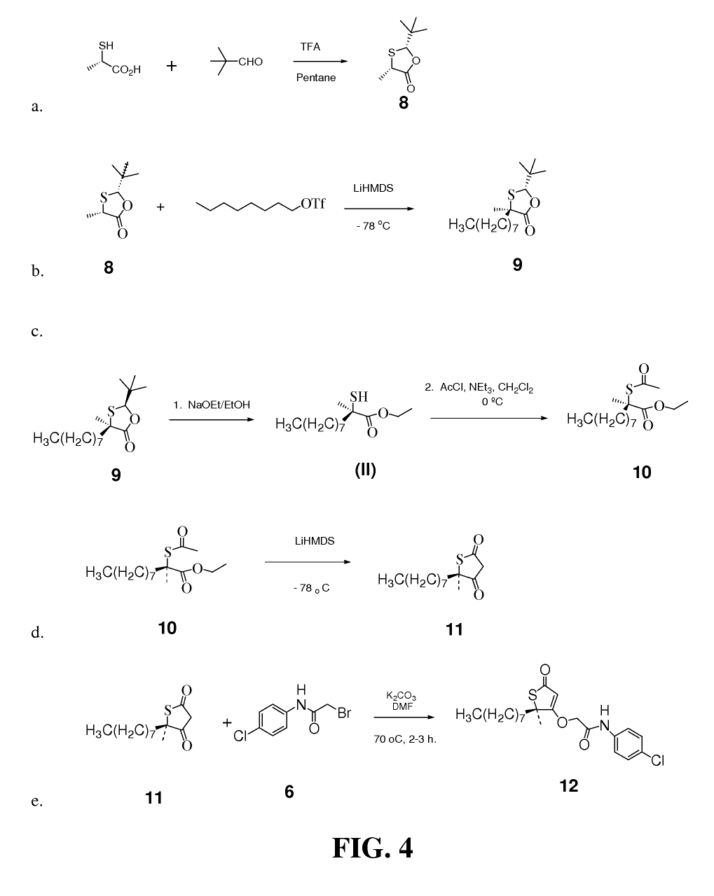

### Synthesis of R-Enantiomer—as Illustrated in FIG. 4

Step A—(S)-2-tert-Butyl-4-methyl-[1,3]oxathiolan-5-one (8). To a flame dried flask under Ar atmosphere was charged with (S)-thiolactic acid (4.17 g, 39.3 mmol), followed by pentane (80 mL) and pivaladehyde (4.48 mL, 41.3 mmol) and few drops of trifluoroacetic acid. The reaction was fitted with Dean-stark apparatus to remove the water. The solution was then heated to reflux for 48 h (55° C.) while removing the water continuously. After cooling to room temperature, the solvent was evaporated completely. The crude product was then recrystallized from pentane:Ether (5:1) at −78° C. The white solid material was filtered thro crucible to give the product 8² (3.23 g, 47.3% yield). ¹H NMR (500 MHz, CDCl₃) δ 1.00 (s, 9H), 1.54 (d, J=7.0 Hz, 3H), 3.94 (q, J=6.5 Hz, 1H), 5.17 (s, 1H). [α]_{D}²⁵ −41.6 (c 1.13, CHCl₃).

Step B—(R)-2-tert-Butyl-4-methyl-4-octa-1,3,5,7-tetraynyl-[1,3]oxathiolan-5-one (3). To a mixture of LiHMDS (16.0 mL, 16.0 mmol, 1 M in THF) in THF (47 mL) at −78° C. was added 8 (2.42 g, 13.9 mmol) in THF (15 mL) drop wise by cannula, and the resulting yellow solution stirred for 30 min at −78° C. Then, octyl triflate 2 (3.85 g, 14.6 mmol) in pentane (8 mL) was added slowly at room temperature via cannula to the solution of the enolate at −78° C. After stifling at −78° C. for 2 h, 1 N HCl (200 mL) was added and the solution was extracted with Et₂O (3×75 mL). The combined organics were dried (MgSO₄), filtered and evaporated. Flash chromatography (2% EtOAc/hexanes) gave pure 9 (2.54 g, 64%). ¹H NMR (500 MHz, CDCl₃ δ 0.86 (t, J=7.0 Hz, 3H), 0.99 (s, 9H), 1.26 (m, 10H), 1.36 (m, 1H), 1.53 (s, 4H), 1.72 (dt, J=4.0, 11.0 Hz, 1H), 1.83 (dt, J=3.5, 13.0 Hz, 1H), 5.12 (s, 1H). [α]_{D}²⁵ +42.1 (c 2.77, CHCl₃)

Step C—(R)-2-Acetylsulfanyl-2-methyl-deca-3,5,7,9-tetraynoic acid ethyl ester (10): To 9 (1.43 g, 5.0 mmol) in EtOH (anhydrous, 14.6 mL) was added NaOEt (12.5 mmol) [freshly prepared from Na metal (300 mg, 12.5 mmol) in EtOH (15 mL)] and the solution was allowed to stir at room temperature. After 30 min, the solution was poured into NH₄Cl₍ₛₐₜ₎/1 N HCl (25 mL, 3:2) and extracted with Et₂O (3×25 mL). The combined organics were then washed thoroughly with H₂O, dried (MgSO₄), filtered, evaporated to give intermediate (II), which was then re-dissolved in CH₂Cl₂ (25 mL). To this pre-cooled solution (0° C.) was added NEt₃ (0.83 mL, 6.0 mmol) and acetyl chloride (0.39 mL, 5.5 mmol). After 40 min at 0° C., NH₄Cl₍ₛₐₜ₎ (50 mL) was added and the solution was extracted with CH₂Cl₂ (3×20 mL). The combined organics were dried (MgSO₄), filtered and evaporated. Flash chromatography (5% EtOAc/hexanes) gave pure 10 (1.29 g, 90.0%). ¹H NMR (500 MHz, CDCl₃) δ 0.85 (t, J=7.0 Hz, 3H), 1.24 (m, 15H), 1.60 (s, 3H), 1.73-1.77 (m, 2H), 2.24 (s, 3H), 4.16 (q, J=7.5 Hz, 2 H). [α]_{D}²⁵ +6.83 (c 1.62, CHCl₃).

Step D—(R)-5-Methyl-5-octa-1,3,5,7-tetraynyl-thiophene-2,4-dione (11). To 10 (1.23 g, 4.27 mmol) in THF (15 mL) at −78° C. was added LiHMDS (6.4 mL, 6.4 mmol, 1.0 M in THF) and the solution was allowed to slowly warm over a 2 h period to −5° C. and then kept at −5° C. for an additional 20 min. The solution was then poured into 1 N HCl (20 mL) and extracted with Et₂O (3×20 mL). The combined organics were dried (MgSO₄), filtered and evaporated. Flash chromatography (20% EtOAc/2% CH₃CO₂H/Hexanes) gave 11 (352.0 mg, 34%). ¹H NMR (500 MHz, CDCl₃) (keto-tautomer) δ 0.86 (t, J=8.0 Hz, 3H), 1.26 (m, 11H), 1.49 (m, 1H), 1.63 (s, 3H), 1.80 (m, 1H), 1.94-2.01 (m, 1H), 3.34 (s, 2H); (enol tautomer characteristic peak) 5.27 (s, 1H). [α]_{D}²⁴+6.03 (c 1.44, CHCl₃)

Step E—(R)—N-(4-Chloro-phenyl)-2-(2-methyl-2-octyl-5-oxo-2,5-dihydro-thiophen-3-yloxy)-acetamide (7) (KS-II-62): A 25 mL round bottom flask was charged with (R)-5-Methyl-5-octa-1,3,5,7-tetraynyl-thiophene-2,4-dione 11 (195.0 mg, 0.80 mmol), N-(4-chlorophenyl)-2-bromoacetamide 6 (209.0 mg, 0.85 mmol), potassium carbonate (220.0 mg, 1.6 mmol, flame dried and cooled under nitrogen atmosphere) and DMF (3.0 mL) under nitrogen atmosphere. The mixture was heated at 70° C. for 2-3 h (monitored by TLC). The solid material was filtered off and washed with diethyl ether. The solution was then diluted with ether (30 mL) and washed with water (3×15 mL), washed with saturated aqueous NH₄Cl (2×10 mL) and brine. The organic layer was dried (MgSO₄), filtered and evaporated to give crude product as a semisolid. The crude product was then recrystallized from diethyl ether:hexane (1:1) to give a white powder (basically crashed out). The product was then filtered and washed with ether:hexane (1:1). The filtrate was concentrated and recrystallized again with ether:hexane (1:1) to give white powder. The combined white powder was dried under vacuum to give the product 12 in 63.0% (206.0 g) yield. ¹H NMR (500 MHz, CDCl₃) δ 0.85 (t, J=7.0 Hz, 3 H), 1.23 (m, 11H), 1.56 (m, 1H), 1.74 (s, 4H), 1.89 (m, 2H), 4.55 (s, 2H), 5.41 (s, 1H), 7.32 (d, J=9.0 Hz, 2H), 7.46 (d, J=9.0 Hz, 2H), 7.76 (s, 1H). [α]_{D}²⁵ +8.56 (c 0.98, CHCl₃).

### Example 6

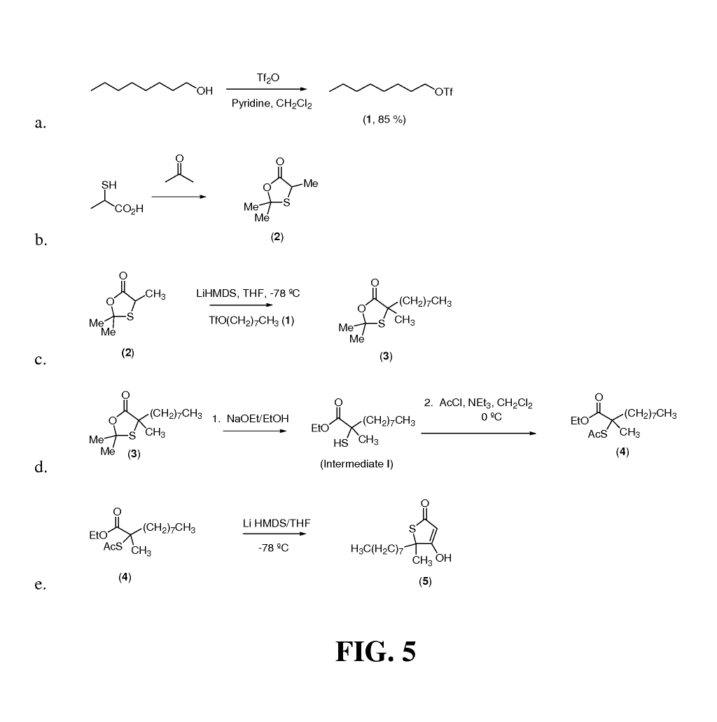

### Alternative Methods for Synthesis of Compounds Bearing O-Acetic Acid Hydrazides—as Illustrated in FIG. 5

Step A—Octyl triflate (1). A dry 3 L 3-necked round bottom flask was fitted with a mechanical stirrer, thermometer and a nitrogen purged inlet. The flask was charged with octanol (150 g, 1.15 mol) in dichloromethane (1050 mL) and cooled to −40° C. followed by the addition of pyridine (107 mL). To the cold solution was added triflic anhydride (209 mL, 1.08 eq) over a period of 45 minutes at −40° C. to −20° C. The reaction was allowed to warm to room temperature. After stirring at room temperature for 1.5 h, the white solid was then filtered through Celite, washed with pentane (2×100 mL). The filtrate was concentrated under reduced pressure at <30° C. to remove most of the solvent. Hot pentane (1,000 mL) was added and this mixture was filtered to remove any remaining pyridine salts. The filtrate was concentrated under reduced pressure at <30° C. to near dryness to afford a clear colorless oil (257.7 g, 85.3%), which was used immediately.

Step B—2,2,4-Trimethyl-[1,3]oxathiolan-5-one (2). A 12 L 3-necked round bottom flask was fitted with a mechanical stirrer, thermometer and Dean-Stark trap under a nitrogen purged atmosphere. The flask was charged with thiolactic acid (1,000 g, 9.4 mol) followed by acetone (12.25 mol, 1.3 eq), p-toluenesulfonic acid (17.9 g, 0.09 mol, 0.01 eq) and benzene (2,400 mL). The mixture was heated to reflux for 47 hours with the continuous removal of water. Approximately 190 mL of water was collected. The solution was cooled to room temperature and diluted with diethyl ether (3,500 mL), washed with 2N Na₂CO₃ (2×2,000 mL) followed by water (2,000 mL) and saturated sodium chloride (2,000 mL). The solution was dried over sulfate, filtered and concentrated under reduced pressure to oil. The crude product was then distilled in vacuo to afford product 2 (967.6 g, 70.2%) as a colorless oil. b.p.=70.5° C.-73° C. (726 mm Hg).

Step C—2,2,4-Trimethyl-4-octyl-[1,3]-oxathiolan-5-one (3). A dry 5 L 3-necked round bottom flask was fitted with a mechanical stirrer, thermometer and a nitrogen purge inlet. To a mixture of LiHMDS (831 mL, 1.0 M in THF) in THF (350 mL) at −78° C. was added drop wise a solution of 2 (110.5 g, 0.76 mol) in tetrahydrofuran (221 mL) over a period of 40 minutes. After stirring the solution at −78° C. for 1 hour, octyl triflate (257.7 g, 0.98 mol, 1.3 eq) was added drop wise over a period of 50 min by maintaining the temperature below −60° C. After stifling at −78° C. for 4 h (monitored by TLC), 2N HCl (800 mL) was added and the solution was extracted with Ethyl acetate (2×600 mL). The combined organic layer was washed with deionized water (3×1,000 mL), dried over magnesium sulfate and filtered. The filtrate was concentrated under reduced pressure to afford a crude oil. The crude product was distilled in vacuo to afford compound 3 (185.9 g, 95.3%) as a colorless oil. b.p.=110° C.-116° C. (726 mm Hg).

Step D—2-Acetylsulfanyl-2-methyl-decanoic acid ethyl ester (4). A 3 L 3-necked round bottom flask was fitted with a mechanical stirrer and a nitrogen purge inlet. To the flask was added ethanol (370 mL) followed by the portion wise addition of sodium metal (21.5 g, 0.93 mol, 1.3 eq). The clear solution was cooled to 20-25° C. followed by the addition of 3 (185 g, 0.72 mol) in ethanol (315 mL). After stifling for 2 h (monitored by TLC), the solution was poured into NH₄Cl₍ₛₐₜ₎/1 N HCl (2,200 mL, 3:2) and extracted with ethyl acetate (2×1,000 mL). The combined organics were then washed thoroughly with H₂O (2×1,000 mL), brine, dried (MgSO₄), filtered, evaporated (182.1 grams of pale yellow oil) and redissolved in CH₂Cl₂ (1,100 mL). To this pre-cooled solution (0° C.) was added NEt₃ (137 g, 1.35 mol) and acetyl chloride (84.3 g, 1.07 mol). After 1 h at 0° C. (monitored by TLC), NH₄Cl₍ₛₐₜ₎ (2,000 mL) was added and the solution was extracted with CH₂Cl₂ (500 mL). The combined organics were washed with water, dried (MgSO₄), filtered and evaporated. The crude product was then purified by vacuum distillation to afford 4 (187.6 g, 90.7%.), b.p.=115° C.-127° C. (726 mm Hg).

Step E—4-Hydroxy-5-methyl-5-octyl-5-H-thiophen-2-one (5). A 6 L 3-necked round bottom flask was fitted with a mechanical stirrer and a nitrogen purge inlet. The flask was charged with 4 (187 g, 0.77 mol) followed by tetrahydrofuran (1,870 mL) and then cooled to −78° C. To the cold solution was added drop wise, LiHMDS (805 mL, 1.24 eq) in tetrahydrofuran over a period of 50 minutes. The reaction mixture was stirred at −70° C. to −50° C. for 1 hour followed by 2 hours at −50° C. to −40° C., 1 hour at −40° C., and then slowly warmed up to room temperature. Reaction was monitored by TLC. The solution was quenched with 2N HCl (1,000 mL) and extracted with ethyl acetate (1,500 mL). Aqueous layer was extracted with 500 mL of ethyl acetate. The combined organic phase was washed with deionized water (2×2,000 mL), dried (MgSO₄), filtered and concentrated under reduced pressure. The crude product was stored in the fridge over night. Crystalline product 5 was isolated (44 g) by filtration and washed with hexane. Filtrate was left in the fridge again without solvent removal. Some more solid was isolated. Operation was repeated until there is no further crystallization. Total isolated yield of 5: 65 g, 41.4%.

### Example 7

### Alternate Purification Process

Once the extraction is done, the organic layer was washed with saturated sodium bicarbonate (twice). The aqueous layer was then acidified with 1N HCl solution (to pH ˜3-4). The aqueous layer was then extracted with ether (3 times), washed with water, brine, dried and concentrated to give the clean product, which was confirmed by NMR.

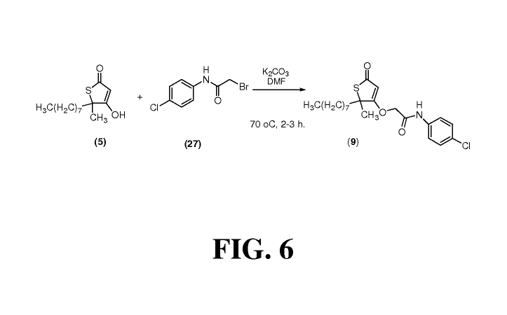

The original organic layer (from the reaction) was washed with water, brine, dried and evaporated to give sulfanyl-2-methyldecanoic acid ethyl ester I. This material was then recycled for the synthesis of compound 4, as set forth in FIG. 6.

### Example 8

### Procedure B for Purification

N-(4-Chloro-phenyl)-2-(2-methyl-2-octyl-5-oxo-2,5-dihydro-thiophen-3-yloxy)-acetamide (9): A 250 mL round bottom flask was charged with 4-hydroxy-5-methyl-5-octyl-5H-thiophen-2-one 5 (9.32 g, 38.5 mmol), N-(4-chlorophenyl)-2-bromoacetamide 27 (9.98 g, 40.4 mmol), potassium carbonate (10.62 g, 77.0 mmol, flame dried and cooled under nitrogen atmosphere) and DMF (96.0 mL) under nitrogen atmosphere. The mixture was heated at 70° C. for 2-3 h (monitored by TLC). The solid material was filtered off and washed with diethyl ether. The solution was then diluted with ether (300 mL) and washed with water (3×100 mL), washed with saturated aqueous NH₄Cl (2×100 mL) and brine. The organic layer was dried (MgSO₄), filtered and evaporated to give crude product as a semisolid. The crude product was then recrystallized from diethyl ether:hexane (1:1) to give a white powder (basically crashed out). The product was then filtered and washed with ether:hexane (1:1). The filtrate was concentrated and recrystallized again with ether:hexane (1:1) to give white powder. The combined white powder was dried under vacuum to give the product 9 in 74% (11.66 g) yield.

### Example 9

### Biological and Biochemical Methods

Compounds according to the invention were subjected to various biological tests as set forth below:

Purification of FAS from ZR-75-1 Human Breast Cancer Cells. Human FAS was purified from cultured ZR-75-1 human breast cancer cells obtained from the American Type Culture Collection. The procedure, adapted from Linn et al., 1981, and Kuhajda et al., 1994, utilizes hypotonic lysis, successive polyethyleneglycol (PEG) precipitations, and anion exchange chromatography. ZR-75-1 cells are cultured at 37° C. with 5% CO₂ in RPMI culture medium with 10% fetal bovine serum, penicillin and streptomycin.

Ten T150 flasks of confluent cells are lysed with 1.5 ml lysis buffer (20 mM Tris-HCl, pH 7.5, 1 mM EDTA, 0.1 mM phenylmethanesulfonyl fluoride (PMSF), 0.1% Igepal CA-630) and bounce homogenized on ice for 20 strokes. The lysate is centrifuged in JA-20 rotor (Beckman) at 20,000 rpm for 30 minutes at 4° C. and the supernatant is brought to 42 ml with lysis buffer. A solution of 50% PEG 8000 in lysis buffer is added slowly to the supernatant to a final concentration of 7.5%. After rocking for 60 minutes at 4° C., the solution is centrifuged in JA-20 rotor (Beckman) at 15,000 rpm for 30 minutes at 4° C. Solid PEG 8000 is then added to the supernatant to a final concentration of 15%. After the rocking and centrifugation is repeated as above, the pellet is resuspended overnight at 4° C. in 10 ml of Buffer A (20 mM K₂HPO₄, pH 7.4). After 0.45 μM filtration, the protein solution is applied to a Mono Q 5/5 anion exchange column (Pharmacia). The column is washed for 15 minutes with buffer A at 1 ml/minute, and bound material is eluted with a linear 60-ml gradient over 60 minutes to 1 M KCl. FAS (MW˜270 kD) typically elutes at 0.25 M KCl in three 0.5 ml fractions identified using 4-15% SDS-PAGE with Coomassie G250 stain (Bio-Rad). FAS protein concentration is determined using the Coomassie Plus Protein Assay Reagent (Pierce) according to manufacturer's specifications using BSA as a standard. This procedure results in substantially pure preparations of FAS (>95%) as judged by Coomassie-stained gels.

Measurement of FAS Enzymatic Activity and Determination of the IC₅₀ of the Compounds FAS activity is measured by monitoring the malonyl-CoA dependent oxidation of NADPH spectrophotometrically at OD₃₄₀ in 96-well plates (Dils et al and Arslanian et al, 1975). Each well contains 2 μg purified FAS, 100 mM K₂HPO₄, pH 6.5, 1 mM dithiothreitol (Sigma), and 187.5 μM β-NADPH (Sigma). Stock solutions of inhibitors are prepared in DMSO at 2, 1, and 0.5 mg/ml resulting in final concentrations of 20, 10, and 5 μg/ml when 1 μl of stock is added per well. For each experiment, cerulenin (Sigma) is run as a positive control along with DMSO controls, inhibitors, and blanks (no FAS enzyme) all in duplicate.

The assay is performed on a Molecular Devices SpectraMax Plus Spectrophotometer. The plate containing FAS, buffers, inhibitors, and controls are placed in the spectrophotometer heated to 37° C. Using the kinetic protocol, the wells are blanked on duplicate wells containing 100 μl of 100 mM K₂HPO₄, pH 6.5 and the plate is read at OD₃₄₀ at 10 sec intervals for 5 minutes to measure any malonyl-CoA independent oxidation of NADPH. The plate is removed from the spectrophotometer and malonyl-CoA (67.4 μM, final concentration per well) and alkynyl-CoA (61.8 μM, final concentration per well) are added to each well except to the blanks. The plate is read again as above with the kinetic protocol to measure the malonyl-CoA dependent NADPH oxidation. The difference between the A OD₃₄₀ for the malonyl-CoA dependent and non-malonyl-CoA dependent NADPH oxidation is the specific FAS activity. Because of the purity of the FAS preparation, non-malonyl-CoA dependent NADPH oxidation is negligible.

The IC₅₀ for the compounds against FAS is determined by plotting the Δ OD₃₄₀ for each inhibitor concentration tested, performing linear regression and computing the best-fit line, r² values, and 95% confidence intervals. The concentration of compound yielding 50% inhibition of FAS is the IC₅₀. Graphs of Δ OD₃₄₀ versus time are plotted by the SOFTmax PRO software (Molecular Devices) for each compound concentration. Computation of linear regression, best-fit line, r², and 95% confidence intervals are calculated using Prism Version 3.0 (Graph Pad Software).

Measurement of [¹⁴C]acetate Incorporation into Total Lipids and Determination of IC₅₀ of Compounds. This assay measures the incorporation of [¹⁴C]acetate into total lipids and is a measure of fatty acid synthesis pathway activity in vitro. It is utilized to measure inhibition of fatty acid synthesis in vitro.

MCF-7 human breast cancer cells cultured as above, are plated at 5×10⁴ cells per well in 24-well plates. Following overnight incubation, the compounds to be tested, solubilized in DMSO, are added at 5, 10, and 20 μg/ml in triplicate, with lower concentrations tested if necessary. DMSO is added to triplicate wells for a vehicle control. C75 is run at 5 and 10 μg/ml in triplicate as positive controls. After 4 hours of incubation, 0.25 μCi of [¹⁴C]acetate (10 μl volume) is added to each well.

After 2 hours of additional incubation, medium is aspirated from the wells and 800 μl of chloroform:methanol (2:1) and 700 μl of 4 mM MgCl₂ is added to each well. Contents of each well are transferred to 1.5 Eppendorf tubes, and spun at full-speed for 2 minutes in a high-speed Eppendorf Microcentrifuge 5415D. After removal of the aqueous (upper) layer, an additional 700 μl of chloroform:methanol (2:1) and 500 μl of 4 mM MgCl₂ are added to each tube and then centrifuged for 1 minutes as above. The aqueous layer is removed with a Pasteur pipette and discarded. An additional 400 μl of chloroform:methanol (2:1) and 200 μl of 4 mM MgCl₂ are added to each tube, then centrifuged and aqueous layer is discarded. The lower (organic) phase is transferred into a scintillation vial and dried at 40° C. under N₂ gas. Once dried, 3 ml of scintillant (APB #NBC5104) is added and vials are counted for ¹⁴C. The Beckman Scintillation counter calculates the average cpm values for triplicates.

The IC₅₀ for the compounds is defined as the concentration of drug leading to a 50% reduction in [¹⁴C]acetate incorporation into lipids compared to controls. This is determined by plotting the average cpm for each inhibitor concentration tested, performing linear regression and computing the best-fit line, r² values, and 95% confidence intervals. The average cpm values are computed by the Beckman scintillation counter (Model LS6500) for each compound concentration. Computation of linear regression, best-fit line, r², and 95% confidence intervals are calculated using Prism Version 3.0 (Graph Pad Software).

Measurement of Fatty Acid Oxidation and Determination of SC₁₅₀ of Compounds This assay measures the degradation of [¹⁴C]palmitate into acid soluble products and is a measure of fatty acid oxidation pathway activity in vitro. It is utilized to measure fatty acid oxidation in vitro.

MCF-7 human breast cancer cells cultured as above, are plated at 2.5×10⁵ cells per well in 24-well plates. Following overnight incubation, the compounds to be tested, solubilized in DMSO, are added at 0.98, 0.39, 1.56, 6.25, 25, and 100 μg/ml in triplicate, with lower concentrations tested if necessary. DMSO is added to triplicate wells for a vehicle control. C75 is run at 5 and 10 μg/ml in triplicate as positive controls. After 1 hour of incubation, medium is removed 100 uM of [¹⁴C] palmitate in cyclodextran and 200 uM carnitine in serum free medium (250 μl volume) is added to each well.

After 30 minutes of additional incubation, the reaction is stopped by addition of 2.6N HClO₄. Contents of each well are transferred to 1.5 ml Eppendorf tubes and 4N KOH is added. The tubes are incubated for 30 minutes at 60° C. 1 M NaAcetate and 3N H2SO4 is added to each tube and vortexed. The tubes are centrifuged at 1000 rpm for 5 minutes at RT. 250 μl of the supernatant is transferred to a 2 ml eppendorf tube. To each tube is added: 938 μl of chloroform:methanol (1:1), 468 μl chloroform and 281 μl of deionized water. The tubes are vortexed and centrifuged at 1000 rpm for 5 minutes at RT. 750 μl of the upper phase is transferred into a scintillation vial 5 ml of scintillant is added and vials are counted for 1 minute for ¹⁴C. The Beckman Scintillation counter calculates the average cpm values for triplicates.

The SC₁₅₀ for the compounds is defined as the concentration of drug leading to a 150% increase in production of acid soluble products of [¹⁴C] palmitate as compared to untreated controls. This is determined by plotting the average cpm for each inhibitor concentration tested, performing linear regression and computing the best-fit line, r² values, and 95% confidence intervals. The average cpm values are computed by the Beckman scintillation counter (Model LS6500) for each compound concentration. Computation of linear regression, best-fit line, r², and 95% confidence intervals are calculated using Prism Version 3.0 (Graph Pad Software). If a compound fails to achieve this 150% threshold it is considered negative. The maximum value achieved is also reported (FAO Max).

XTT Cytotoxicity Assay The XTT assay is a non-radioactive alternative for the [⁵¹Cr] release cytotoxicity assay. XTT is a tetrazolium salt that is reduced to a formazan dye only by metabolically active, viable cells. The reduction of XTT is measured spectrophotometrically as OD₄₉₀-OD₆₅₀.

To measure the cytotoxicity of specific compounds against cancer cells, 9×10³ MCF-7 human breast cancer cells (shown in the tables as “(M)”), obtained from the American Type Culture Collection are plated per well in 96 well plates in DMEM medium with 10% fetal bovine serum, insulin, penicillin, and streptomycin. Following overnight culture at 37° C. and 5% CO₂, the compounds to be tested, dissolved in DMSO, are added to the wells in 1 μl volume at the following concentrations: 80, 40, 20, 10, 5, 2.5, 1.25, and 0.625 μg/ml in triplicate. Additional concentrations are tested if required. 1 μl of DMSO is added to triplicate wells are the vehicle control. C75 is run at 40, 20, 10, 15, 12.5, 10, and 5 μg/ml in triplicate as positive controls.

After 72 hours of incubation, cells are incubated for 4 hours with the XTT reagent as per manufacturer's instructions (Cell Proliferation Kit II (XTT) Roche). Plates are read at OD₄₉₀ and OD₆₅₀ on a Molecular Devices SpectraMax Plus Spectrophotometer. Three wells containing the XTT reagent without cells serve as the plate blank. XTT data are reported as OD₄₉₀-OD₆₅₀. Averages and standard error of the mean are computed using SOFTmax Pro software (Molecular Dynamics).

The IC₅₀ for the compounds is defined as the concentration of drug leading to a 50% reduction in OD₄₉₀-OD₆₅₀ compared to controls. The OD₄₉₀-OD₆₅₀ are computed by the SOFTmax PRO software (Molecular Devices) for each compound concentration. IC₅₀ is calculated by linear regression, plotting the FAS activity as percent of control versus drug concentrations. Linear regression, best-fit line, r², and 95% confidence intervals are determined using Prism Version 3.0 (Graph Pad Software).

The test was also run against OVCAR3 cells (“OV”), and HCT116 cells (“H”).

Weight Loss Screen Balb/C mice (Jackson Labs) are utilized for the initial weight loss screening. Animals are housed in temperature and 12 hour day/night cycle rooms and fed mouse chow and water ad lib. Three mice are utilized for each compound tested with vehicle controls in triplicate per experiment. For the experiments, mice are housed separately for each compound tested three mice to a cage. Compounds are diluted in DMSO at 10 mg/ml and mice are injected intraperitoneally with 60 mg/kg in approximately 100 μl of DMSO or with vehicle alone. Mice are observed and weighed daily; average weights and standard errors are computed with Excel (Microsoft). The experiment continues until treated animals reach their pretreatment weights.

Antimicrobial Properties A broth microdilution assay is used to assess the antimicrobial activity of the compounds. Compounds are tested at twofold serial dilutions, and the concentration that inhibits visible growth (OD₆₀₀ at 10% of control) is defined as the MIC. Microorganisms tested include Staphylococcus aureus (ATCC # 29213), Enterococcus faecalis (ATCC # 29212), Pseudomonas aerpginosa (ATCC # 27853), and Escherichia coli (ATCC # 25922). The assay is performed in two growth media, Mueller Hinton Broth and Trypticase Soy Broth.

A blood (Tsoy/5% sheep blood) agar plate is inoculated from frozen stocks maintained in T soy broth containing 10% glycerol and incubated overnight at 37° C. Colonies are suspended in sterile broth so that the turbidity matches the turbidity of a 0.5 McFarland standard. The inoculum is diluted 1:10 in sterile broth (Mueller Hinton or Trypticase soy) and 195 μl is dispensed per well of a 96-well plate. The compounds to be tested, dissolved in DMSO, are added to the wells in 5 μl volume at the following concentrations: 25, 12.5, 6.25, 3.125, 1.56 and 0.78 μg/ml in duplicate. Additional concentrations are tested if required. 5 μl of DMSO added to duplicate wells are the vehicle control. Serial dilutions of positive control compounds, vancomycin (E. faecalis and S. aureus) and tobramycin (E. coli and P. aerpginosa), are included in each run.

After 24 hours of incubation at 37° C., plates are read at OD₆₀₀ on a Molecular Devices SpectraMax Plus Spectrophotometer. Average OD₆₀₀ values are computed using SOFTmax Pro Software (Molecular Devices) and MIC values are determined by linear regression analysis using Prism version 3.02 (Graph Pad Software, San Diego). The MIC is defined as the concentration of compound required to produce an OD₆₀₀ reading equivalent to 10% of the vehicle control reading.

Results of the Biological Testing

| | | | |
|---|---|---|---|
| FAS (IC₅₀) | ¹⁴C (IC₅₀) | XTT (IC₅₀) | XTT (IC₅₀) |

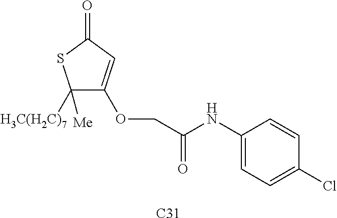

| Limited by Solubility | 19.2 ± 2.0 μg/ml | 5.2 ± 2.0 μg/ml (M) | 5.9 ± 2.7 μg/ml (H) |
|---|---|---|---|
| 109 μg/ml (SB) | | 9.2 ± 5.0 μg/ml (OV) | |
| | Weight Loss | | |
| | 60 mg/kg: 0.2% (day 1) | | |
| FAO SC 150 | FAO Max | | |
| Neg | 106% at 1.56 μg/ml | | |
| SA/MH (MIC) | SA/Tsoy (MIC) | EF/MH | EF/Tsoy (MIC) |
| 6 μg/ml | 3 μg/ml | Neg | 44 μg/ml |

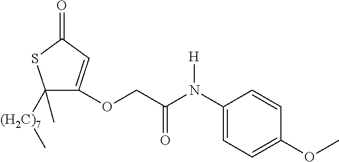

| (SB) | 23.0 μg/ml | 9.7 μg/ml (M) | 15.6 μg/ml (H) |
|---|---|---|---|
| | | 17.8 μg/ml (OV) | |
| CPT I Stim | Weight Loss | | |
| Not Tested | Not Tested | | |
| FAO SC 150 | FAO Max | | GPAT IC₅₀ |
| Neg | 90% at | | Not Tested |
| | 0.098 μg/ml | | |
| SA/MH (MIC) | SA/Tsoy (MIC) | EF/MH (MIC) | EF/Tsoy (MIC) |
| μg/ml | μg/ml | Neg | Neg |

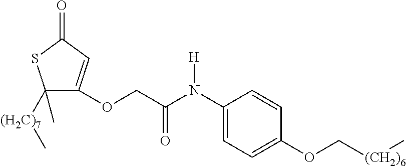

| (SB) | Neg (>80 μg/ml) | >80 μg/ml (M) | >80 μg/ml (H) |
|---|---|---|---|
| | | 67.0 μg/ml (OV) | |
| CPT I Stim | Weight Loss | | |
| Not Tested | Not Tested | | |
| FAO SC 150 | FAO Max | | GPAT IC₅₀ |
| Neg | 97% at | | Not Tested |
| | 0.098 μg/ml | | |
| SA/MH (MIC) | SA/Tsoy (MIC) | EF/MH (MIC) | EF/Tsoy (MIC) |
| μg/ml | μg/ml | Neg | Neg |

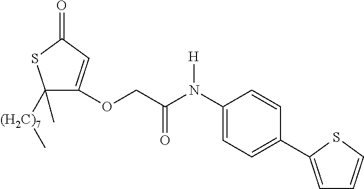

| (SB) | 80.2 μg/ml | >80 μg/ml (M) | >80 μg/ml (H) |
|---|---|---|---|
| | | >80 μg/ml (OV) | |
| CPT I Stim | Weight Loss | | |
| Not Tested | Not Tested | | |
| FAO SC 150 | FAO Max | | GPAT IC₅₀ |
| 61.9 μg/ml | 168% at | | Not Tested |
| | 100 μg/ml | | |
| SA/MH (MIC) | SA/Tsoy (MIC) | EF/MH (MIC) | EF/Tsoy (MIC) |
| μg/ml | μg/ml | Neg | Neg |

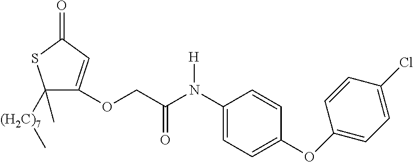

| (SB) | Neg (>80 μg/ml) | 3.1 μg/ml (M) | 6.3 μg/ml (H) |
|---|---|---|---|
| | | 5.0 μg/ml (OV) | |
| CPT I Stim | Weight Loss | | |
| Not Tested | 60 mg/kg: 3.1% (day 4) | | |
| FAO SC 150 | FAO Max | | GPAT IC₅₀ |
| Neg | 109% at | | Not Tested |
| | 6.25 μg/ml | | |
| SA/MH (MIC) | SA/Tsoy (MIC) | EF/MH (MIC) | EF/Tsoy (MIC) |
| μg/ml | μg/ml | Neg | Neg |

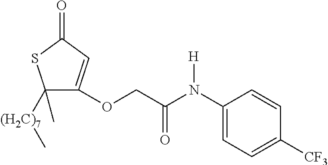

| (SB) | <2.5 μg/ml | 2.2 μg/ml (M) | 4.8 μg/ml (H) |
|---|---|---|---|
| | Repeat at lower | 4.0 μg/ml (OV) | |
| CPT I Stim | Weight Loss | | |
| Not Tested | Not Tested | | |
| FAO SC 150 | FAO Max | | GPAT IC₅₀ |
| Neg | 130% at | | Not Tested |
| | 6.25 μg/ml | | |
| SA/MH (MIC) | SA/Tsoy (MIC) | EF/MH (MIC) | EF/Tsoy (MIC) |
| μg/ml | μg/ml | Neg | Neg |

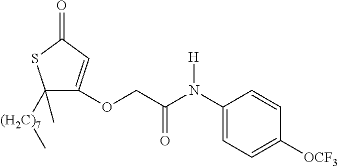

| (SB) | 8.2 μg/ml | 1.8 μg/ml (M) | 3.5 μg/ml (H) |
|---|---|---|---|
| | | 3.3 μg/ml (OV) | |
| CPT I Stim | Weight Loss | | |
| Not Tested | 60 mg/kg: 2.2% (day 1) | | |
| FAO SC 150 | FAO Max | | GPAT IC₅₀ |
| μg/ml | % at | | Not Tested |
| | μg/ml | | |
| SA/MH (MIC) | SA/Tsoy (MIC) | EF/MH (MIC) | EF/Tsoy (MIC) |
| μg/ml | μg/ml | Neg | Neg |

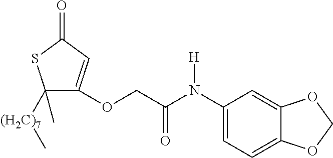

| (SB) | Not Tested | 8.2 μg/ml (M) | 14.8 μg/ml (H) |
|---|---|---|---|
| | | 9.3 μg/ml (OV) | |
| CPT I Stim | Weight Loss | | |
| Not Tested | Not Tested | | |
| FAO SC 150 | FAO Max | | GPAT IC₅₀ |
| Neg | 45% at | | Not Tested |
| | 0.098 μg/ml | | |
| SA/MH (MIC) | SA/Tsoy (MIC) | EF/MH (MIC) | EF/Tsoy (MIC) |
| μg/ml | μg/ml | Neg | Neg |

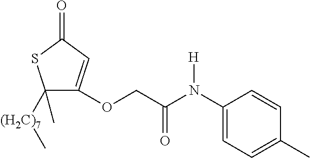

| (SB) | Not Tested | 6.8 μg/ml (M) | 12.8 μg/ml (H) |
|---|---|---|---|
| | | 8.1 μg/ml (OV) | |
| CPT I Stim | Weight Loss | | |
| Not Tested | Not Tested | | |
| FAO SC 150 | FAO Max | | GPAT IC₅₀ |
| μg/ml | % at | | Not Tested |
| | μg/ml | | |
| SA/MH (MIC) | SA/Tsoy (MIC) | EF/MH (MIC) | EF/Tsoy (MIC) |
| μg/ml | μg/ml | Neg | Neg |

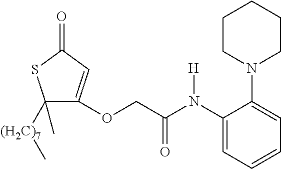

| (SB) | Not Tested | 18.6 μg/ml (M) | 13.1 μg/ml (H) |
|---|---|---|---|
| | | 15.5 μg/ml (OV) | |
| CPT I Stim | Weight Loss | | |
| Not Tested | Not Tested | | |
| FAO SC 150 | FAO Max | | GPAT IC₅₀ |
| Neg | 119% at | | Not Tested |
| | 1.56 μg/ml | | |
| SA/MH (MIC) | SA/Tsoy (MIC) | EF/MH (MIC) | EF/Tsoy (MIC) |
| μg/ml | μg/ml | Neg | Neg |

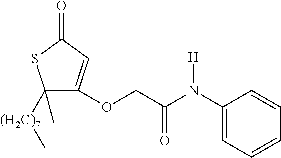

| (SB) | Not Tested | 6.2 μg/ml (M) | 7.1 μg/ml (H) |
|---|---|---|---|
| | | 12.1 μg/ml (OV) | |
| CPT I Stim | Weight Loss | | |
| Not Tested | Not Tested | | |
| FAO SC 150 | FAO Max | | GPAT IC₅₀ |
| Neg | 122% at | | Not Tested |
| | 0.098 μg/ml | | |
| SA/MH (MIC) | SA/Tsoy (MIC) | EF/MH (MIC) | EF/Tsoy (MIC) |
| μg/ml | μg/ml | Neg | Neg |

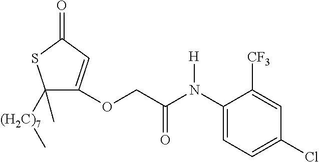

| (SB) | Not Tested | 9.6 μg/ml (M) | 14.0 μg/ml (H) |
|---|---|---|---|
| | | 24.0 μg/ml (OV) | |
| CPT I Stim | Weight Loss | | |
| Not Tested | Not Tested | | |
| FAO SC 150 | FAO Max | | GPAT IC₅₀ |
| 1.9 μg/ml | 141% at | | Not Tested |
| | 1.56 μg/ml | | |
| SA/MH (MIC) | SA/Tsoy (MIC) | EF/MH (MIC) | EF/Tsoy (MIC) |
| μg/ml | μg/ml | Neg | Neg |

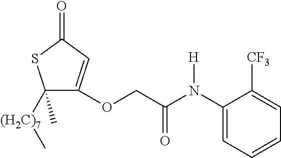

| (SB) | >80 μg/ml | 17.6 μg/ml (M) | 15.6 μg/ml (H) |
|---|---|---|---|
| | Sol Prob 80 μg/ml | 24.1 μg/ml (OV) | |
| CPT I Stim | Weight Loss | | |
| Not Tested | Not Tested | | |
| FAO SC 150 | FAO Max | | GPAT IC₅₀ |
| Neg | 105% at | | Not Tested |
| | 1.56 μg/ml | | |
| SA/MH (MIC) | SA/Tsoy (MIC) | EF/MH (MIC) | EF/Tsoy (MIC) |
| μg/ml | μg/ml | μg/ml | μg/ml |

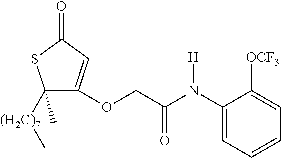

| (SB) | >80 μg/ml | >80 μg/ml (M) | 78.8 μg/ml (H) |
|---|---|---|---|
| | | >80 μg/ml (OV) | |
| CPT I Stim | Weight Loss | | |
| Not Tested | Not Tested | | |
| FAO SC 150 | FAO Max | | GPAT IC₅₀ |
| Neg | 116% at | | Not Tested |
| | 25 μg/ml | | |
| SA/MH (MIC) | SA/Tsoy (MIC) | EF/MH (MIC) | EF/Tsoy (MIC) |
| μg/ml | μg/ml | μg/ml | μg/ml |

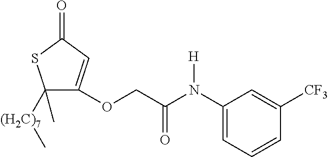

| (SB) | 12.3 μg/ml | 5.9 μg/ml (M) | 7.6 μg/ml (H) |
|---|---|---|---|
| | Sol Prob 80 μg/ml | 11.0 μg/ml (OV) | |
| CPT I Stim | Weight Loss | | |
| Not Tested | Not Tested | | |
| FAO SC 150 | FAO Max | | GPAT IC₅₀ |
| Neg | 75% at | | Not Tested |
| | 0.395 μg/ml | | |
| SA/MH (MIC) | SA/Tsoy (MIC) | EF/MH (MIC) | EF/Tsoy (MIC) |
| μg/ml | μg/ml | μg/ml | μg/ml |

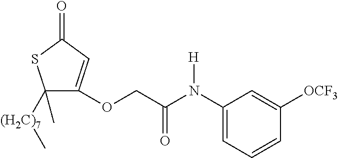

| (SB) | 17.1 μg/ml | 6.4 μg/ml (M) | 8.0 μg/ml (H) |
|---|---|---|---|
| | Sol Prob 40 μg/ml | 11.6 μg/ml (OV) | |
| CPT I Stim | Weight Loss | | |
| Not Tested | Not Tested | | |
| FAO SC 150 | FAO Max | | GPAT IC₅₀ |
| Neg | 122% at | | Not Tested |
| | 1.56 μg/ml | | |
| SA/MH (MIC) | SA/Tsoy (MIC) | EF/MH (MIC) | EF/Tsoy (MIC) |
| μg/ml | μg/ml | μg/ml | μg/ml |

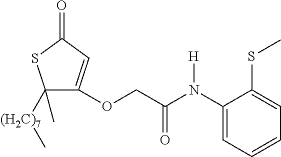

| (SB) | >80 μg/ml | 26.9 μg/ml (M) | 31.4 μg/ml (H) |
|---|---|---|---|
| | Sol Prob 40 μg/ml | 43.8 μg/ml (OV) | |
| CPT I Stim | Weight Loss | | |
| Not Tested | Not Tested | | |
| FAO SC 150 | FAO Max | | GPAT IC₅₀ |
| Neg | 114% at | | Not Tested |
| | 6.25 μg/ml | | |
| SA/MH (MIC) | SA/Tsoy (MIC) | EF/MH (MIC) | EF/Tsoy (MIC) |
| μg/ml | μg/ml | μg/ml | μg/ml |

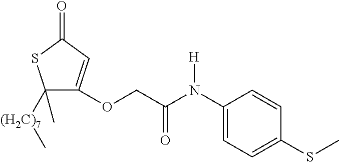

| (SB) | >80 μg/ml | 7.9 μg/ml (M) | 11.5 μg/ml (H) |
|---|---|---|---|
| | Sol Prob 40 μg/ml | 16.9 μg/ml (OV) | |
| CPT I Stim | Weight Loss | | |
| Not Tested | Not Tested | | |
| FAO SC 150 | FAO Max | | GPAT IC₅₀ |
| Neg | 100% at | | Not Tested |
| | 6.25 μg/ml | | |
| SA/MH (MIC) | SA/Tsoy (MIC) | EF/MH (MIC) | EF/Tsoy (MIC) |
| μg/ml | μg/ml | μg/ml | μg/ml |

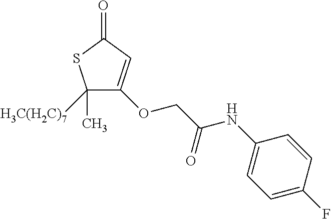

| | Not Tested | 6.5 μg/ml (M) | 5.6 μg/ml (H) |
|---|---|---|---|
| μg/ml (SB) | Sol Prob 80 | 11.1 ml (OV) | |
| CPT I Stim | Weight Loss | | |
| Not Tested | 60 mg/ml: 2.4% (day 1) | | |
| FAO SI 150 | FAO Max | | |
| Neg | 106% at 1.56 μg/ml | | |
| SA/MH (MIC) | SA/Tsoy (MIC) | EF/MH (MIC) | EF/Tsoy (MIC) |
| μg/ml | μg/ml | μg/ml | μg/ml |

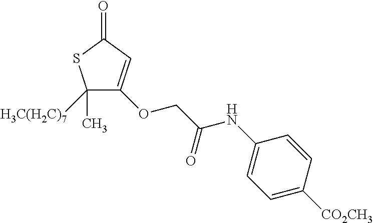

| | Not Tested | 6.5 μg/ml (M) | 6.3 μg/ml (H) |
|---|---|---|---|
| μg/ml (SB) | Sol prob 80 | 12.7 ml (OV) | |
| CPT I Stim | Weight Loss | | |
| Not Tested | Not Tested | | |
| FAO SI 150 | FAO Max | | |
| Neg | 126% at 6.25 μg/ml | | |
| SA/MH (MIC) | SA/Tsoy (MIC) | EF/MH (MIC) | EF/Tsoy (MIC) |
| μg/ml | μg/ml | μg/ml | μg/ml |

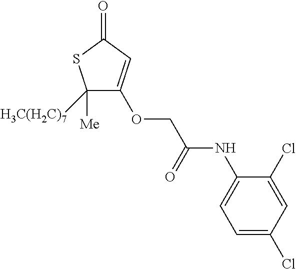

| Neg (SB) | Not Tested | 16.8 μg/ml (M) | 13.1 μg/ml (H) |
|---|---|---|---|
| Solubility Prob 40 μg/ml | | 64.5 ml (OV) | |
| CPT I Stim | Weight Loss | | |
| Not Tested | Not Tested | | |
| FAO SC 150 | FAO Max | | |
| Neg | 141% at | | |
| | 1.56 μg/ml | | |
| SA/MH (MIC) | SA/Tsoy (MIC) | EF/MH (MIC) | EF/Tsoy (MIC) |
| μg/ml | μg/ml | μg/ml | μg/ml |

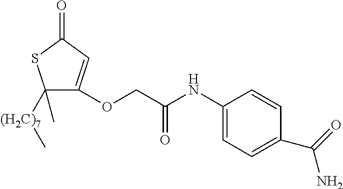

| (SB) | 12.3 ug/ml | 10.2 ug/ml (M) | 10.6 ug/ml (H) |
|---|---|---|---|
| | Sol Prob 80 ug/ml | 21.5 ug/ml (OV) | |
| CPT I Stim | Weight Loss | | |
| Not Tested | Not Tested | | |
| FAO SC 150 | FAO Max | | GPAT IC₅₀ |
| | % at | | Not Tested |
| | ug/ml | | |
| SA/MH (MIC) | SA/Tsoy (MIC) | EF/MH (MIC) | EF/Tsoy (MIC) |
| ug/ml | ug/ml | ug/ml | ug/ml |

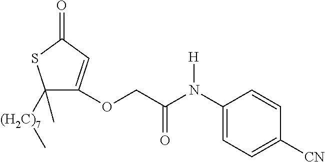

| (SB) | >80 ug/ml | 3.8 ug/ml (M) | 5.3 ug/ml (H) |
|---|---|---|---|
| | Sol Prob 80 ug/ml | 6.6 ug/ml (OV) | |
| CPT I Stim | Weight Loss | | |
| Not Tested | Not Tested | | |
| FAO SC 150 | FAO Max | | GPAT IC₅₀ |
| ug/ml | % at | | Not Tested |
| | ug/ml | | |
| SA/MH (MIC) | SA/Tsoy (MIC) | EF/MH (MIC) | EF/Tsoy (MIC) |
| ug/ml | ug/ml | ug/ml | ug/ml |

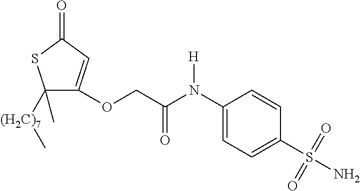

| (SB) | 26.3 ug/ml | 7.0 ug/ml (M) | 8.7 ug/ml (H) |
|---|---|---|---|
| | Sol Prob 80 ug/ml | 13.4 ug/ml (OV) | |
| CPT I Stim | Weight Loss | | |
| Not Tested | Not Tested | | |
| FAO SC 150 | FAO Max | | GPAT IC₅₀ |
| ug/ml | % at | | Not Tested |
| | ug/ml | | |
| SA/MH (MIC) | SA/Tsoy (MIC) | EF/MH (MIC) | EF/Tsoy (MIC) |
| ug/ml | ug/ml | ug/ml | ug/ml |

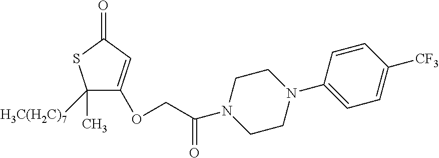

| (SB) | | 50.7 ug/ml (M) | >80 ug/ml (H) |
|---|---|---|---|
| | | >80 ug/ml (OV) | |
| CPT I Stim | Weight Loss | | |
| Not Tested | Not Tested | | |
| FAO SC 150 | FAO Max | | GPAT IC₅₀ |
| ug/ml | 118% at | | Not Tested |
| | 1.56 ug/ml | | |
| SA/MH | SA/Tsoy (MIC) | EF/MH (MIC) | EF/Tsoy (MIC) |
| ug/ml | ug/ml | ug/ml | ug/ml |

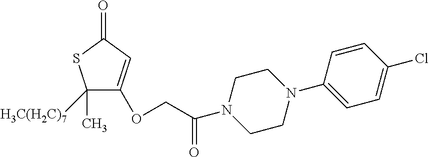

| (SB) | | 35.7 ug/ml (M) | 9.7 ug/ml (H) |
|---|---|---|---|
| | | 24.4 ug/ml (OV) | |
| CPT I Stim | Weight Loss | | |
| Not Tested | Not Tested | | |
| FAO SC 150 | FAO Max | | GPAT IC₅₀ |
| ug/ml | 79% at | | Not Tested |
| | 0.098 ug/ml | | |
| SA/MH | SA/Tsoy (MIC) | EF/MH (MIC) | EF/Tsoy (MIC) |
| ug/ml | ug/ml | ug/ml | ug/ml |

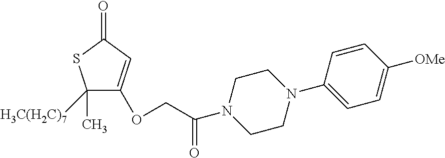

| (SB) | | >80 ug/ml (M) | >80 ug/ml (H) |
|---|---|---|---|
| | | >80 ug/ml (OV) | |
| CPT I Stim | Weight Loss | | |
| Not Tested | Not Tested | | |
| FAO SC 150 | FAO Max | | GPAT IC₅₀ |
| ug/ml | 53% at | | Not Tested |
| | 0.098 ug/ml | | |
| SA/MH | SA/Tsoy (MIC) | EF/MH (MIC) | EF/Tsoy (MIC) |
| ug/ml | ug/ml | ug/ml | ug/ml |

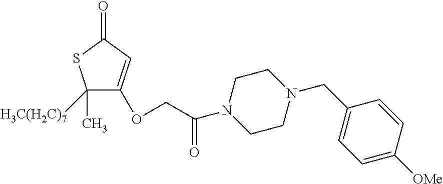

| (SB) | | 13.6 ug/ml (M) | 69.7 ug/ml (H) |
|---|---|---|---|
| | | 79.8 ug/ml (OV) | |
| CPT I Stim | Weight Loss | | |
| Not Tested | Not Tested | | |
| FAO SC 150 | FAO Max | | GPAT IC₅₀ |
| ug/ml | 83% at | | Not Tested |
| | 0.098 ug/ml | | |
| SA/MH | SA/Tsoy (MIC) | EF/MH (MIC) | EF/Tsoy (MIC) |
| ug/ml | ug/ml | ug/ml | ug/ml |

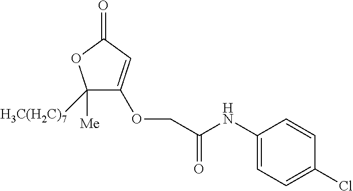

| Neg(SB) to 50 g/ml | Not tested | 6.0 ug/ml (M) | 4.8 ug/ml (H) |
|---|---|---|---|
| May be limited by | | 9.2 ug/ml (OV) | |
| Solubility 80 μg/ml | | | |
| CPT I Stim | Weight Loss | | |
| Not Tested | Not Tested | | |
| FAO SC 150 | FAO Max | | GPAT IC₅₀ |
| Neg | 95% at 0.39 μg/ml1 | | Not Tested |
| | 0.098 ug/ml | | |
| SA/MH (MIC) | SA/Tsoy (MIC) | EF/MH | EF/Tsoy (MIC) |
| 6 ug/ml | 3 ug/ml | 70 ug/ml | Neg |

1. A compound comprising the formula: 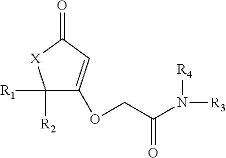
wherein X is a heteroatom selected from the group consisting of O, S, and N;
R¹ and R² are independently selected from the group consisting of H, C₁-C₂₀ alkyl, cycloalkyl, alkenyl, aryl, arylalkyl, and alkylaryl; and
R³ and R⁴ are independently a hydrogen or a member of a substituted or unsubstituted ring having 4-6 carbon atoms, provided that both R³ and R⁴ are not hydrogens and further that, if neither R³ and R⁴ are hydrogens, then R³ and R⁴ are members of the same substituted or unsubstituted ring having 4-6 carbon atoms.

2-77. (canceled)

78. The compound of claim 1, wherein X is either an oxygen or sulfur.

79. The compound of claim 1, wherein R³ is a hydrogen and R⁴ is selected from the group consisting of a substituted or unsubstituted aryl group, a substituted or unsubstituted heteroaryl group, and a substituted or unsubstituted heterocyclic ring group each having 4-6 carbon atoms.

80. The compound of claim 79, wherein R⁴ is substituted with one or more of a first substituent group selected from the group consisting of a halogen atom, a C₁-C₃ alkyl group, a C₁-C₃ haloalkyl group, —OR⁵, —SR⁵, —CN, —CONH₂, —SO₂NH₂, —C(O)OR⁶—CONHR⁷ and a cycloalkyl or a heterocyclic ring, wherein the cycloalkyl or heterocyclic ring of the first substituent group is optionally aromatic, is optionally fused to two adjacent atoms of R⁴, and is optionally substituted with at least one substituent group comprised of R⁵,
wherein R⁵ is selected from the group consisting of a C₁-C₈ alkyl, C₁-C₈ alkoxy, aryl, alkylaryl, and arylalkyl, and is optionally substituted with one or more of a second substituent group selected from the group consisting of a halogen atom, a C₁-C₃ alkyl group, a C₁-C₃ alkoxy group, a C₁-C₃ haloalkyl group, and a C₁-C₃ haloalkoxy group,
wherein R⁶ is comprised of a C₁-C₈ alkyl group and R⁷ is selected from the group consisting of a C₁-C₈ alkyl group, an allyl group, a morpholine, a piperazine, an N-substituted piperazine with R⁵, and a 5- or 6-membered heterocycle containing N, O, S or any combination thereof.

81. The compound of claim 80, wherein R³ is a hydrogen and R⁴ is an aryl group optionally substituted with one or more of the first substituent group.

82. The compound of claim 1, wherein R¹ is a straight or branched chain C₆-C₈ alkyl group.

83. The compound of claim 1, wherein R¹ is a straight or branched chain C₈ alkyl group.

84. The compound of claim 1, wherein R² is a straight or branched chain C₁-C₃ alkyl group.

85. The compound of claim 1, wherein R² is a methyl group.

86. A compound comprising the formula: 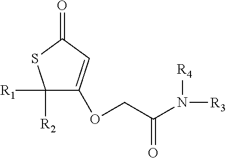
wherein R¹ and R² are independently selected from the group consisting of H, C₁-C₂₀ alkyl, cycloalkyl, alkenyl, aryl, arylalkyl, and alkylaryl; and
R³ and R⁴ are independently a hydrogen or a member of a substituted or unsubstituted ring having 4-6 carbon atoms, provided that both R³ and R⁴ are not a hydrogen and further that, if neither R³ and R⁴ are hydrogens, then R³ and R⁴ are members of the same substituted or unsubstituted ring having 4-6 carbon atoms.

87. A compound of claim 86, wherein said compound is selected from the group consisting of

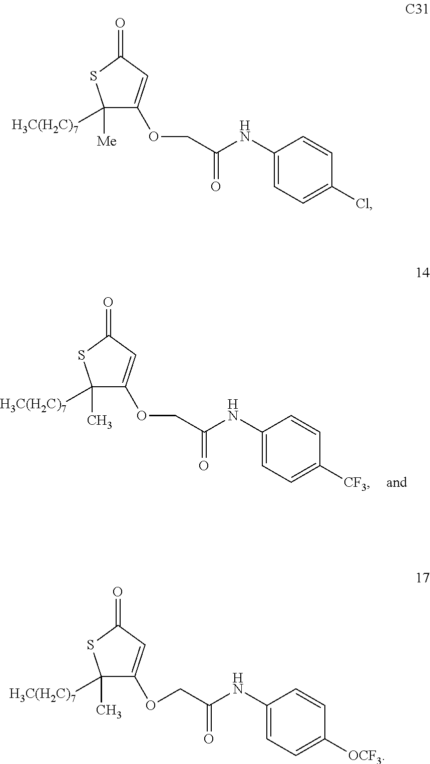

88. A pharmaceutical composition comprising a pharmaceutical diluent and a compound according to claim 1.

89. The pharmaceutical composition of claim 88, wherein X is sulfur.

90. The pharmaceutical composition of claim 88, wherein R¹ is a straight or branched chain C₆-C₈ alkyl group and R² is a straight or branched chain C₁-C₃ alkyl.

91. The pharmaceutical composition of claim 88, wherein R³ is a hydrogen and R⁴ is an aryl group optionally substituted with one or more of the first substitution group.

92. The pharmaceutical composition of claim 89, wherein the compound is selected from the group consisting of

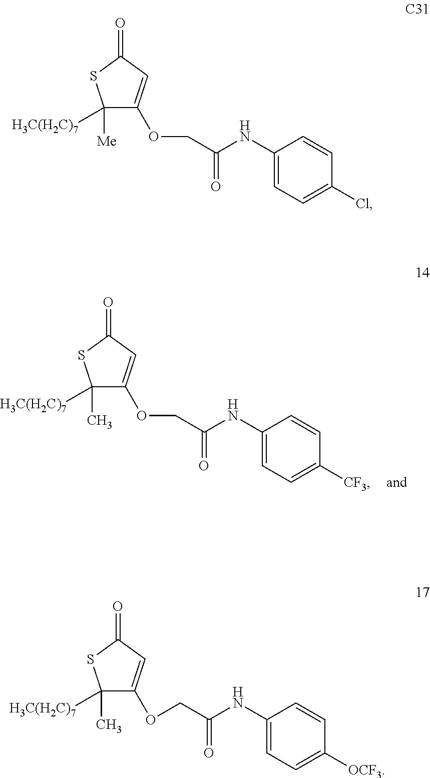

93. A method of treating cancer, inducing weight loss, inhibiting growth of invasive microbial cells, or inhibiting fatty acid synthase activity in a subject, comprising administering to the subject an effective amount of a pharmaceutical composition according to claim 88.

94. The method of claim 93, wherein the method comprises treating cancer.

95. The method of claim 93, wherein the subject is an animal.

96. The method of claim 95, wherein the subject is a human.

97. The method of claim 93, wherein X is sulfur.

98. The method of claim 93, wherein R¹ is a straight or branched chain C₆-C₈ alkyl group and R² is a straight or branched chain C₁-C₃ alkyl.

99. The method of claim 93, wherein R³ is a hydrogen and R⁴ is an aryl group optionally substituted with one or more of the first substituent group.

100. The method of claim 93, wherein the pharmaceutical composition includes one or more compounds selected from the group consisting of: [chemical structure]
